SEQUENCE LISTING (40 sequences)

SEQ ID NO: 1 - DNA, 23 bp, synthetic construct
  source 1..23: mol_type = other DNA
gaccaggaactaatcagacaagg

SEQ ID NO: 2 - DNA, 19 bp, synthetic construct
  source 1..19: mol_type = other DNA
tcaaccacgttcccgaagg

SEQ ID NO: 3 - DNA, 25 bp, synthetic construct
  source 1..25: mol_type = other DNA
cgacattccgaagaacgctgaagcg

SEQ ID NO: 4 - DNA, 25 bp, synthetic construct
  source 1..25: mol_type = other DNA
gccatcaaattggatgacaaagatc

SEQ ID NO: 5 - DNA, 21 bp, synthetic construct
  source 1..21: mol_type = other DNA
taggctctgttggtgggaatg

SEQ ID NO: 6 - DNA, 27 bp, synthetic construct
  source 1..27: mol_type = other DNA
cattttgctgaataagcatattgacgc

SEQ ID NO: 7 - DNA, 22 bp, synthetic construct
  source 1..22: mol_type = other DNA
aatcartgtgtaacattaggga

SEQ ID NO: 8 - DNA, 20 bp, synthetic construct
  source 1..20: mol_type = other DNA
aggcwgctctccctarcatt

SEQ ID NO: 9 - DNA, 21 bp, synthetic construct
  source 1..21: mol_type = other DNA
cgcggagtacgatcgagkgta

SEQ ID NO: 10 - DNA, 24 bp, synthetic construct
  source 1..24: mol_type = other DNA
aagtattragtgaratggtcatgt

SEQ ID NO: 11 - DNA, 24 bp, synthetic construct
  source 1..24: mol_type = other DNA
ggcaatyttrttaccatcagtwga

SEQ ID NO: 12 - DNA, 26 bp, synthetic construct
  source 1..26: mol_type = other DNA
gatgccacaactgcttatgctaatag

SEQ ID NO: 13 - DNA, 19 bp, synthetic construct
  source 1..19: mol_type = other DNA
cccctcaaagccgagatcg

SEQ ID NO: 14 - DNA, 17 bp, synthetic construct
  source 1..17: mol_type = other DNA
ggcacggtgagcgtgaa

SEQ ID NO: 15 - DNA, 21 bp, synthetic construct
  source 1..21: mol_type = other DNA
atggctaaagacaagaccaat

SEQ ID NO: 16 - DNA, 22 bp, synthetic construct
  source 1..22: mol_type = other DNA
aaggcaaagcagaactagcaga

SEQ ID NO: 17 - DNA, 24 bp, synthetic construct
  source 1..24: mol_type = other DNA
cagatagaggcaccaattagtgct

SEQ ID NO: 18 - DNA, 20 bp, synthetic construct
  source 1..20: mol_type = other DNA
acactgttggttyggtggga

SEQ ID NO: 19 - DNA, 19 bp, synthetic construct
  source 1..19: mol_type = other DNA
agatttggacctgcgagcg

SEQ ID NO: 20 - DNA, 20 bp, synthetic construct
  source 1..20: mol_type = other DNA
gagcggctgtctccacaagt

SEQ ID NO: 21 - DNA, 23 bp, synthetic construct
  source 1..23: mol_type = other DNA
ttctgacctgaaggctctgcgcg

SEQ ID NO: 22 - DNA, 23 bp, synthetic construct
  source 1..23: mol_type = other DNA
gaccaggaattaatcagacaagg

SEQ ID NO: 23 - DNA, 23 bp, synthetic construct
  source 1..23: mol_type = other DNA
gaccatgaactaatcagacaagg

SEQ ID NO: 24 - DNA, 23 bp, synthetic construct
  source 1..23: mol_type = other DNA
gaccaggaactaaccagacaagg

SEQ ID NO: 25 - DNA, 19 bp, synthetic construct
  source 1..19: mol_type = other DNA
tcaaccacgttcccaaagg

SEQ ID NO: 26 - DNA, 19 bp, synthetic construct
  source 1..19: mol_type = other DNA
tcaaccacgttccagaagg

SEQ ID NO: 27 - DNA, 19 bp, synthetic construct
  source 1..19: mol_type = other DNA
taaaccacgttcccgaagg

SEQ ID NO: 28 - DNA, 19 bp, synthetic construct
  source 1..19: mol_type = other DNA
tcaaccacgttcccgaaag

SEQ ID NO: 29 - DNA, 19 bp, synthetic construct
  source 1..19: mol_type = other DNA
tcaaccacgttaccgaagg

SEQ ID NO: 30 - DNA, 25 bp, synthetic construct
  source 1..25: mol_type = other DNA
cgacatttcgaagaacgctgaagcg

SEQ ID NO: 31 - DNA, 25 bp, synthetic construct
  source 1..25: mol_type = other DNA
agacattccgaagaacgctgaagcg

SEQ ID NO: 32 - DNA, 25 bp, synthetic construct
  source 1..25: mol_type = other DNA
gccatcaaattggatgacaaagatt

SEQ ID NO: 33 - DNA, 25 bp, synthetic construct
  source 1..25: mol_type = other DNA
gccatcaaatttgatgacaaagatc

SEQ ID NO: 34 - DNA, 25 bp, synthetic construct
  source 1..25: mol_type = other DNA
gtcatcaaattggatgacaaagatc

SEQ ID NO: 35 - DNA, 25 bp, synthetic construct
  source 1..25: mol_type = other DNA
gccatcaaattggatgacaaagttc

SEQ ID NO: 36 - DNA, 21 bp, synthetic construct
  source 1..21: mol_type = other DNA
taggctctgttggtggggatg

SEQ ID NO: 37 - DNA, 21 bp, synthetic construct
  source 1..21: mol_type = other DNA
taggttctgttggtgggaatg

SEQ ID NO: 38 - DNA, 21 bp, synthetic construct
  source 1..21: mol_type = other DNA
taggctctgttggtggaaatg

SEQ ID NO: 39 - DNA, 21 bp, synthetic construct
  source 1..21: mol_type = other DNA
taggctctgttgatgggaatg

SEQ ID NO: 40 - DNA, 27 bp, synthetic construct
  source 1..27: mol_type = other DNA
cgttttgctgaataagcatattgacgc

(19) United States
(12) Patent Application Publication
(10) Pub. No.: US 2023/0332254 A1
(43) Pub. Date: Oct. 19, 2023

(54) COMPOSITIONS AND METHODS FOR RAPID DETECTION OF SARS-CoV-2

(71) Applicant: THE TRUSTEES OF COLUMBIA UNIVERSITY IN THE CITY OF NEW YORK, New York, NY (US)

(72) Inventors: Walter Ian Lipkin, New York, NY (US); Thomas BRIESE, White Plains, NY (US); Nischay MISHRA, New York, NY (US); Rafal TOKARZ, Queens Village, NY (US); Simon H. WILLIAMS, New York, NY (US)

(21) Appl. No.: 17/881,008

(22) Filed: Aug. 4, 2022

Related U.S. Application Data
(63) Continuation of application No. PCT/US2021/019075, filed on Feb. 22, 2021.
(60) Provisional application No. 62/978,980, filed on Feb. 20, 2020.
(60) Provisional application No. 63/009,126, filed on Apr. 13, 2020.
(60) Provisional application No. 63/056,229, filed on Jul. 24, 2020.

(51) Int. Cl. C12Q 1/70; C12Q 1/6844

(52) U.S. Cl. CPC C12Q 1/701; C12Q 1/6844; C12Q 2600/16

(57) ABSTRACT

Disclosed here are compositions and methods for the detection of at least one virus viruses using a one-step assay. The viruses to be detected include at least SARS-CoV-2 and influenza viruses. In particular, the disclosure relates to a method of detection of the viruses using specific primers and probes designed to detect and if necessary differentiate between the viruses.

### CROSS REFERENCE TO RELATED APPLICATION

The present application is a continuation of PCT Application No. PCT/US2021/019075, filed Feb. 22, 2021, which claims priority to U.S. Patent Applications Ser. Nos. 62/978,980 filed Feb. 20, 2020, 63/009,126 filed Apr. 13, 2020, and 63/056,229 filed Jul. 24, 2020, all of which are hereby incorporated by reference in their entirety.

### SEQUENCE LISTING

The instant application contains a Sequence Listing which has been submitted electronically in XML file format and is hereby incorporated by reference in its entirety. Said XML copy, created on Sep. 20, 2022, is named 01001_008490-US3_SEtxt and is 36,329 bytes in size.

### FIELD

This disclosure relates to compositions and methods for the detection of at least one virus using a one-step assay. The viruses to be detected includes at least SARS-CoV-2. In some embodiments, the viruses to be detected include at least SARS-CoV-2 and and influenza viruses. In particular, the disclosure relates to a method of detection of the viruses using specific primers and probes designed to detect and if necessary differentiate between the viruses.

### BACKGROUND

In December 2019, a cluster of cases were found postive for severe pneumonia-like illness in Wuhan, Hubei Province, China. In first week of January 2020, novel coronavirus (2019-nCoV) was discovered and identified in oral swab/nasopharengeal swab samples obtained from the viral pneumonia cases. On Feb. 11, 2020, the virus was named SARS-CoV-2 by the International Committee on Taxonomy of Viruses (ICTV). On Jan. 30, 2020, the World Health Organization declared the outbreak of SARS-CoV-2 a global health emergency. On Feb. 4, 2020, the Secretary of the Department of Health and Human Services (HHS), USA also declared a public health emergency with significant potential to affect national security or the health and security of United States citizens living abroad. Over the past year, SARS-CoV-2 has quickly spread within and outside of Hubei Province and was introduced to additional countries.

After the full genome was released on Jan. 7, 2020 several laboratories created a qualitative real time PCR assay to detect SARS-CoV-2. The United States Center for Disease Control (CDC) also reported a SARS-CoV-2 realtime PCR assay and obtained Emergency User Authorisation for the assay from the Food and Drug Administration on Feb. 4, 2020.

It is unknown how well those assays perform with clinical materials because there are no reference panels for validation. What is known, however, is that PCR of human nasopharyngeal aspirates has not successfully identified many outbreak victims with known disease, thus, screening now includes computerized tomography (CT) for evidence of pneumonia. CT is expensive, labor intensive, and exposes patients to radiation. Furthermore, CT cannot be used for analysis of feces, blood, or environmental surveillance. Along with SARS-CoV-2, seasonal influenza A and B also pose a risk of respiratory illness and pneumonia. Rapid, sensitive, specific, affordable, simultaneous and differential diagnosis of SARS-CoV-2, influenza A and influenza B virus with appropriate reaction control is urgently needed.

### SUMMARY

The current disclosure provides for an assay, a multiplex one-step reverse transcription real time polymerase chain reaction test intended for the detection and diagnosis of SARS-CoV-2 virus infection by detecting viral RNA in specimens. The assay can also be used for the differential detection of RNA from SARS-CoV-2, and influenza A and B in samples, thus differentially detecting the viruses. SARS-CoV-2 RNA is typically detectable in nasopharyngeal and oropharyngeal aspirate during the acute phase of infection and up to two weeks following onset of symptoms. Positive results with the disclosed assay are indicative of acute viral infection.

The current disclosure provides compositions, methods, and kits for detecting the presence of nucleic acids of certain viruses specifically SARS-CoV-2. Additionally, the current disclosure allows for the differential detection of certain viruses. In particular, the current disclosure allows for the differential detection of SARS-CoV-2 virus as well as influenza A and/or B virus in a one-step assay using a polymerase chain reaction format. The disclosed method and assay is rapid, inexpensive, sensitive, and specific, and allows for the detection and diagnosis of SARS-CoV-2 virus, as well as influenza A and/or B virus. In certain embodiments, the current disclosure allows the detection and the determination of which specific virus or viruses are found in a single sample. In one aspect, the disclosure provides primers and probes that can not only detect the viruses in a single sample, but differentiate which virus or viruses are contained in a single sample.

In certain aspects, the disclosure provides a method for detecting a nucleic acid of SARS-CoV-2 virus in a one-step assay, i.e., a single sample. In some embodiments, the methods comprise amplifying the nucleic acid of the viruses in the presence of a detectably-labeled oligonucleotide probe. In some embodiments, the methods comprise amplifying the nucleic acid of the viruses using a primer, wherein the primer comprises SEQ ID NOs: 1, 2, 4, 5, 7, 8, 10, or 11. In some embodiments, the methods comprise amplifying the nucleic acid of the virus using more than one primer, in primer pair or groups, wherein the primer pairs or groups comprise: SEQ ID NO: 1 and SEQ ID NO: 2; SEQ ID NO: 4 and SEQ ID NO: 5; SEQ ID NO: 7 and SEQ ID NO: 8; and SEQ ID NO: 10 and SEQ ID NO: 11. In some embodiments, the oligonucleotide probe comprises SEQ ID NOs: 3, 6, 9, or 12. In some embodiments, the methods comprise amplifying the nucleic acid of the virus using more than one primer, in primer pair or groups, and detecting the presence of the nucleic acids with a detectably-labeled probe, wherein the primer pairs or groups and probes comprise: SEQ ID NO: 1, SEQ ID NO: 2, and SEQ ID NO: 3; SEQ ID NO: 4, SEQ ID NO: 5, and SEQ ID NO: 6; SEQ ID NO: 7, SEQ ID NO: 8, and SEQ ID NO. 9; and SEQ ID NO: 10, SEQ ID NO: 11, and SEQ ID NO: 12. In some embodiments of the method, all of the primer groups and probes are used to detect the nucleic acid of the virus in one sample at the same time, i.e., simultaneously. In some embodiments of the method, all of the primer groups and probes are used to detect the nucleic acid of the virus in one sample at consecutive times, i.e., concurrently. In some embodiments, the nucleic acid to be detected is RNA. In some embodiments, the nucleic acid to be detected is cDNA.

In further aspects, the disclosure provides a method for detecting a nucleic acid of SARS-CoV-2 virus in a one-step assay, i.e., a single sample. In some embodiments, the methods comprise using primers and probes which target two different regions of the nucleocapsid gene of SARS-CoV-2. In some embodiments, the methods comprise amplifying the nucleic acid of the viruses in the presence of a detectably-labeled oligonucleotide probe. In some embodiments, the methods comprise amplifying the nucleic acid of the viruses using a primer, wherein the primer comprises SEQ ID NOs: 1, 2, 4, or 5. In some embodiments, the methods comprise amplifying the nucleic acid of the virus using more than one primer, in primer pair or groups, wherein the primer pairs or groups comprise: SEQ ID NO: 1 and SEQ ID NO: 2; and SEQ ID NO: 4 and SEQ ID NO: 5. In some embodiments, the oligonucleotide probe comprises SEQ ID NOs: 3, or 6. In some embodiments, the methods comprise amplifying the nucleic acid of the virus using more than one primer, in primer pair or groups, and detecting the presence of the nucleic acids with a detectably-labeled probe, wherein the primer pairs or groups and probes comprise: SEQ ID NO: 1, SEQ ID NO: 2, and SEQ ID NO: 3; and SEQ ID NO: 4, SEQ ID NO: 5, and SEQ ID NO: 6. In some embodiments of the method, all of the primer groups and probes are used to detect the nucleic acid of the virus in one sample at the same time, i.e., simultaneously. In some embodiments of the method, all of the primer groups and probes are used to detect the nucleic acid of the virus in one sample at consecutive times, i.e., concurrently. In some embodiments, the nucleic acid to be detected is RNA. In some embodiments, the nucleic acid to be detected is cDNA.

In further aspects, the disclosure provides a method for detecting a nucleic acid of SARS-CoV-2 virus and/or influenza A and/or B viruses in a one-step assay, i.e., a single sample. In some embodiments, the methods comprise amplifying the nucleic acid of the viruses in the presence of a detectably-labeled oligonucleotide probe. In some embodiments, the methods comprise amplifying the nucleic acid of the viruses using a primer, wherein the primer comprises SEQ ID NOs: 1, 2, 4, 5, 7, 8, 10, 11, 13, 14, 16, or 17. In some embodiments, the methods comprise amplifying the nucleic acid of the viruses using more than one primer, in primer pair or groups, wherein the primer pairs or groups comprise: SEQ ID NO: 1 and SEQ ID NO: 2; SEQ ID NO: 4 and SEQ ID NO: 5; SEQ ID NO: 7 and SEQ ID NO: 8; SEQ ID NO: 10 and SEQ ID NO: 11; SEQ ID NO: 13 and SEQ ID NO: 14; and SEQ ID NO: 16 and SEQ ID NO: 17. In some embodiments, the oligonucleotide probe comprises SEQ ID NOs: 3, 6, 9, 12, 15, or 18. In some embodiments, the methods comprise amplifying the nucleic acid of the viruses using more than one primer, in primer pair or groups, and detecting the presence of the nucleic acids with a detectably-labeled probe, wherein the primer pairs or groups and probes comprise: SEQ ID NO: 1, SEQ ID NO: 2, and SEQ ID NO: 3; SEQ ID NO: 4, SEQ ID NO: 5, and SEQ ID NO: 6; SEQ ID NO: 7, SEQ ID NO: 8, and SEQ ID NO. 9; SEQ ID NO: 10, SEQ ID NO: 11, and SEQ ID NO: 12; SEQ ID NO: 13, SEQ ID NO: 14, and SEQ ID NO: 15; and SEQ ID NO: 16, SEQ ID NO: 17, and SEQ ID NO: 19. In some embodiments, the methods comprise amplifying the nucleic acid of the viruses using more than one primer, in primer pair or groups, and detecting the presence of the nucleic acids with a detectably-labeled probe, wherein the primer pairs or groups and probes comprise: SEQ ID NO: 1, SEQ ID NO: 2, and SEQ ID NO: 3; SEQ ID NO: 13, SEQ ID NO: 14, and SEQ ID NO: 15; and SEQ ID NO: 16, SEQ ID NO: 17, and SEQ ID NO: 19. In some embodiments of the method, all of the listed primer groups and probes are used to detect the nucleic acid of the viruses in one sample at the same time, i.e., simultaneously. In some embodiments of the method, all of the listed primer groups and probes are used to detect the nucleic acid of the viruses in one sample at consecutive times, i.e., concurrently. In some embodiments, the nucleic acid to be detected is RNA. In some embodiments, the nucleic acid to be detected is cDNA.

The present disclosure also provides methods of detecting SARS-CoV-2 virus in a one-step assay, i.e., a single sample. In some embodiments, the methods comprise amplifying a nucleic acid of SARS-CoV-2 virus with at least one oligonucleotide comprising a sequence selected from the group consisting of SEQ ID NO: 1, SEQ ID NO: 2, SEQ ID NO: 4, SEQ ID NO: 5, SEQ ID NO: 7, SEQ ID NO: 8, SEQ ID NO: 10, and SEQ ID NO:11, under conditions to allow for initiation of amplification of at least part of the nucleotide sequence from the oligonucleotide; and detecting the amplified nucleic acid, thereby detecting the virus. In some embodiments, the methods comprise amplifying the nucleic acid of the virus using more than one primer, in primer pair or groups, wherein the primer pairs or groups comprise: SEQ ID NO: 1 and SEQ ID NO: 2; SEQ ID NO: 4 and SEQ ID NO: 5; SEQ ID NO: 7 and SEQ ID NO: 8; and SEQ ID NO: 10 and SEQ ID NO: 11. In some embodiments, the oligonucleotide probe comprises SEQ ID NOs: 3, 6, 9, or 12. In some embodiments, the methods comprise amplifying the nucleic acid of the virus using more than one primer, in primer pair or groups, and detecting the presence of the nucleic acids with a detectably-labeled probe, wherein the primer pairs or groups and probes comprise: SEQ ID NO: 1, SEQ ID NO: 2, and SEQ ID NO: 3; SEQ ID NO: 4, SEQ ID NO: 5, and SEQ ID NO: 6; SEQ ID NO: 7, SEQ ID NO: 8, and SEQ ID NO. 9; and SEQ ID NO: 10, SEQ ID NO: 11, and SEQ ID NO: 12. In some embodiments of the method, all of the primer groups and probes are used to detect the nucleic acid of the viruses in one sample at the same time, i.e., simultaneously. In some embodiments of the method, all of the primer groups and probes are used to detect the nucleic acid of the viruses in one sample at consecutive times, i.e., concurrently. In some embodiments, the nucleic acid is RNA. In some embodiments, the nucleic acid is cDNA.

The present disclosure also provides methods of detecting SARS-CoV-2 virus in a one-step assay, i.e., a single sample. In some embodiments, the methods comprise using primers and probes which target two different regions of the nucleocapsid gene of SARS-CoV-2. In some embodiments, the methods comprise amplifying a nucleic acid of SARS-CoV-2 virus with at least one oligonucleotide comprising a sequence selected from the group consisting of SEQ ID NO: 1, SEQ ID NO: 2, SEQ ID NO: 4, and SEQ ID NO: 5, under conditions to allow for initiation of amplification of at least part of the nucleotide sequence from the oligonucleotide; and detecting the amplified nucleic acid, thereby detecting the virus. In some embodiments, the methods comprise amplifying the nucleic acid of the virus using more than one primer, in primer pair or groups, wherein the primer pairs or groups comprise: SEQ ID NO: 1 and SEQ ID NO: 2; and SEQ ID NO: 4 and SEQ ID NO: 5. In some embodiments, the oligonucleotide probe comprises SEQ ID NOs: 3, or 6. In some embodiments, the methods comprise amplifying the nucleic acid of the virus using more than one primer, in primer pair or groups, and detecting the presence of the nucleic acids with a detectably-labeled probe, wherein the primer pairs or groups and probes comprise: SEQ ID NO: 1, SEQ ID NO: 2, and SEQ ID NO: 3; and SEQ ID NO: 4, SEQ ID NO: 5, and SEQ ID NO: 6. In some embodiments of the method, all of the primer groups and probes are used to detect the nucleic acid of the viruses in one sample at the same time, i.e., simultaneously. In some embodiments of the method, all of the primer groups and probes are used to detect the nucleic acid of the viruses in one sample at consecutive times, i.e., concurrently. In some embodiments, the nucleic acid is RNA. In some embodiments, the nucleic acid is cDNA.

In further aspects, the disclosure provides a method for detecting SARS-CoV-2 virus and/or influenza A and/or B viruses in a one-step assay, i.e., a single sample. In some embodiments, the methods comprise amplifying the nucleic acid of the viruses in the presence of a detectably-labeled oligonucleotide probe. In some embodiments, the methods comprise amplifying the nucleic acid of the viruses using a primer, wherein the primer comprises SEQ ID NOs: 1, 2, 4, 5, 7, 8, 10, 11, 13, 14, 16, or 17. In some embodiments, the methods comprise amplifying the nucleic acid of the viruses using more than one primer, in primer pair or groups, wherein the primer pairs or groups comprise: SEQ ID NO: 1 and SEQ ID NO: 2; SEQ ID NO: 4 and SEQ ID NO: 5; SEQ ID NO: 7 and SEQ ID NO: 8; SEQ ID NO: 10 and SEQ ID NO: 11; SEQ ID NO: 13 and SEQ ID NO: 14; and SEQ ID NO: 16 and SEQ ID NO: 17. In some embodiments, the oligonucleotide probe comprises SEQ ID NOs: 3, 6, 9, 12, 15, or 18. In some embodiments, the methods comprise amplifying the nucleic acid of the viruses using more than one primer, in primer pair or groups, and detecting the presence of the nucleic acids with a detectably-labeled probe, wherein the primer pairs or groups and probes comprise: SEQ ID NO: 1, SEQ ID NO: 2, and SEQ ID NO: 3; SEQ ID NO: 4, SEQ ID NO: 5, and SEQ ID NO: 6; SEQ ID NO: 7, SEQ ID NO: 8, and SEQ ID NO. 9; SEQ ID NO: 10, SEQ ID NO: 11, and SEQ ID NO: 12; SEQ ID NO: 13, SEQ ID NO: 14, and SEQ ID NO: 15; and SEQ ID NO: 19, SEQ ID NO: 20, and SEQ ID NO: 21. In some embodiments, the methods comprise amplifying the nucleic acid of the viruses using more than one primer, in primer pair or groups, and detecting the presence of the nucleic acids with a detectably-labeled probe, wherein the primer pairs or groups and probes comprise: SEQ ID NO: 1, SEQ ID NO: 2, and SEQ ID NO: 3; SEQ ID NO: 13, SEQ ID NO: 14, and SEQ ID NO: 15; and SEQ ID NO: 16, SEQ ID NO: 17, and SEQ ID NO: 19. In some embodiments of the method, all of the listed primer groups and probes are used to detect the nucleic acid of the viruses in one sample at the same time, i.e., simultaneously. In some embodiments of the method, all of the listed primer groups and probes are used to detect the nucleic acid of the viruses in one sample at consecutive times, i.e., concurrently. In some embodiments, the nucleic acid to be detected is RNA. In some embodiments, the nucleic acid to be detected is cDNA.

In yet further aspects, the disclosure provides a method for detecting and differentiating SARS-CoV-2 virus from influenza A and/or B viruses in a one-step assay, i.e., a single sample. In some embodiments, the methods comprise amplifying the nucleic acid of the viruses in the presence of a detectably-labeled oligonucleotide probe. In some embodiments, the methods comprise amplifying the nucleic acid of the viruses using a primer, wherein the primer comprises SEQ ID NOs: 1, 2, 4, 5, 7, 8, 10, 11, 13, 14, 16, or 17.

In some embodiments, the methods comprise amplifying the nucleic acid of the viruses using more than one primer, in primer pair or groups, wherein the primer pairs or groups comprise: SEQ ID NO: 1 and SEQ ID NO: 2; SEQ ID NO: 4 and SEQ ID NO: 5; SEQ ID NO: 7 and SEQ ID NO: 8; SEQ ID NO: 10 and SEQ ID NO: 11; SEQ ID NO: 13 and SEQ ID NO: 14; and SEQ ID NO: 16 and SEQ ID NO: 17. In some embodiments, the oligonucleotide probe comprises SEQ ID NOs: 3, 6, 9, 12, 15, or 18. In some embodiments, the methods comprise amplifying the nucleic acid of the viruses using more than one primer, in primer pair or groups, and detecting the presence of the nucleic acids with a detectably-labeled probe, wherein the primer pairs or groups and probes comprise: SEQ ID NO: 1, SEQ ID NO: 2, and SEQ ID NO: 3; SEQ ID NO: 4, SEQ ID NO: 5, and SEQ ID NO: 6; SEQ ID NO: 7, SEQ ID NO: 8, and SEQ ID NO. 9; SEQ ID NO: 10, SEQ ID NO: 11, and SEQ ID NO: 12; SEQ ID NO: 13, SEQ ID NO: 14, and SEQ ID NO: 15; and SEQ ID NO: 19, SEQ ID NO: 20, and SEQ ID NO: 21. In some embodiments, the methods comprise amplifying the nucleic acid of the viruses using more than one primer, in primer pair or groups, and detecting the presence of the nucleic acids with a detectably-labeled probe, wherein the primer pairs or groups and probes comprise: SEQ ID NO: 1, SEQ ID NO: 2, and SEQ ID NO: 3; SEQ ID NO: 13, SEQ ID NO: 14, and SEQ ID NO: 15; and SEQ ID NO: 16, SEQ ID NO: 17, and SEQ ID NO: 19.

In some embodiments of the method, all of the listed primer groups and probes are used to detect the nucleic acid of the viruses in one sample at the same time, i.e., simultaneously. In some embodiments of the method, all of the listed primer groups and probes are used to detect the nucleic acid of the viruses in one sample at consecutive times, i.e., concurrently. In some embodiments, the nucleic acid to be detected is RNA. In some embodiments, the nucleic acid to be detected is cDNA.

In certain embodiments of the foregoing, the probe comprises a detectable moiety. The detectable moiety can be any detectable moiety known to one of skill in the art without limitation. For example, the detectable moiety can be a fluorescent moiety. In certain embodiments, the fluorescent moiety can be selected from the group consisting of fluorescein-family dyes, polyhalofluorescein-family dyes, hexachlorofluorescein-family dyes, coumarin-family dyes, rhodamine-family dyes, cyanine-family dyes, oxazine-family dyes, thiazine-family dyes, squaraine-family dyes, chelated lanthanide-family dyes, and BODIPY®-family dyes.

In certain embodiments of the foregoing, the probe comprises a quencher moiety. The quencher moiety can be any quencher moiety known to one of skill in the art without limitation. In certain embodiments, the quencher moiety can be selected from the group consisting of fluorescein-family dyes, polyhalofluorescein-family dyes, hexachlorofluorescein-family dyes, coumarin-family dyes, rhodamine-family dyes, cyanine-family dyes, oxazine-family dyes, thiazine-family dyes, squaraine-family dyes, chelated lanthanide-family dyes, BODIPY®-family dyes, and non-fluorescent quencher moieties. In certain embodiments, the non-fluorescent quencher moieties can be BHQT™-family dyes, Iowa Black™m, or Dabcyl.

In certain embodiments of the foregoing, the methods comprise amplifying the nucleic acid of the viruses in the presence of a detectably-labeled nucleic acid probe which comprises a fluorescent moiety and a quencher moiety. In certain embodiments, fragmentation of the detectably-labeled probe by a template-dependent nucleic acid polymerase with 5′-3′ nuclease activity separates the fluorescent moiety from the quencher moiety. In certain embodiments, the fragmentation of the probe and thus the presence of the nucleic acid of the virus can be detected by monitoring emission of fluorescence.

In certain embodiments of the foregoing methods, primers and probes for human RNase are also used (SEQ ID NOs: 19-21).

In addition to the foregoing methods, the present invention further provides nucleic acid primers and probes for detecting a nucleic acid of the SARS-CoV-2 virus and/or influenza A and/or B viruses.

In certain embodiments, the disclosure provides for a nucleic acid primer for detecting the nucleoprotein (N) gene of the SARS-CoV-2 virus comprising SEQ ID NO: 1 and/or SEQ ID NO: 2. In certain embodiments, the disclosure provides for a nucleic acid primer for detecting a different region of the N gene of the SARS-CoV-2 virus comprising SEQ ID NO: 4 and/or SEQ ID NO: 5. In certain embodiments, the disclosure provides for a nucleic acid primer for detecting the 3′ UTR of SARS-CoV-2 comprising SEQ ID NO: 7 and/or SEQ ID NO: 8. In certain embodiments, the disclosure provides for a nucleic acid primer for detecting the ORF1 Ab of SARS-CoV-2 virus comprising SEQ ID NO: 10 and/or SEQ ID NO: 11.

In certain embodiments, the disclosure provides for a nucleic acid primer for detecting the matrix gene of influenza A virus comprising SEQ ID NO: 13 and/or SEQ ID NO: 14. In certain embodiments, the disclosure provides for a nucleic acid primer for detecting the matrix gene of influenza B virus comprising SEQ ID NO: 16 and/or SEQ ID NO: 17.

In other aspects, the disclosure provides a nucleic acid probe for detecting the SARS-CoV-2 and/or influenza A and/or B viruses. In certain embodiments, the disclosure provides for a nucleic acid probe for detecting the nucleoprotein (N) gene of the SARS-CoV-2 virus comprising SEQ ID NO: 3. In certain embodiments, the disclosure provides for a nucleic acid probe for detecting a different region of the N gene of the SARS-CoV-2 virus comprising SEQ ID NO: 6. In certain embodiments, the disclosure provides for a nucleic acid probe for detecting the 3′ UTR of SARS-CoV-2 comprising SEQ ID NO: 9. In certain embodiments, the invention provides for a nucleic acid probe for detecting the ORF1 Ab of SARS-CoV-2 virus comprising SEQ ID NO: 12. In certain embodiments, the disclosure provides for a nucleic acid probe for detecting the matrix gene of influenza A virus comprising SEQ ID NO: 15. In certain embodiments, the disclosure provides for a nucleic acid probe for detecting the matrix gene of influenza B virus comprising SEQ ID NO: 18.

In certain embodiments, the disclosure provides a nucleic acid probe comprising a fluorescent moiety and a quencher moiety. In certain embodiments, the fluorescent moiety is positioned relative to the quencher moiety such that a photon emitted by the fluorescent moiety is absorbed by the quencher moiety when the probe is intact. Fragmentation of the probe by an enzyme with 5′ nuclease activity separates the fluorescent moiety from the quencher moiety such that a photon emitted by the fluorescent moiety can be detected.

In other aspects, the disclosure provides a kit for the detection of a nucleic acid of the SARS-CoV-2 virus and/or influenza A and/or B viruses and/or the detection of the SARS-CoV-2 virus and/or influenza A and/or B viruses. In certain embodiments, the kit comprises a combination of one or more of the primers and probes disclosed herein. In certain embodiments the kit comprises one or more primers chosen from the group consisting of SEQ ID NOs: 1, 2, 4, 5, 7, 8, 10, 11, 13, 14, 16, and 17. In certain embodiments, the kit comprises one or more probes chosen from the group consisting of SEQ ID NOs: 3, 6, 9, 12, 15, and 18. In certain embodiments, the kit further comprises primers and probes for positive control sequences. In certain embodiments, the kit further comprises primers and probes for detecting human RNase. In certain embodiments, the kit comprises primers and probe comprising SEQ ID NOs: 19-21.

In certain embodiments, the kit comprises one or more primer pairs or group and probes wherein the primer pairs or groups and probes comprise: SEQ ID NO: 1, SEQ ID NO: 2, and SEQ ID NO: 3; and SEQ ID NO: 4, SEQ ID NO: 5, and SEQ ID NO: 6. In certain embodiments, the kit further comprises primers and probes for detecting human RNase. In certain embodiments, the kit comprises primers and probe comprising SEQ ID NOs: 19-21.

In other embodiments, the kit comprises one or more primer pairs or groups and probes, wherein the primer pair or groups and probes comprise: SEQ ID NO: 1, SEQ ID NO: 2, and SEQ ID NO: 3; SEQ ID NO: 13, SEQ ID NO: 14, and SEQ ID NO: 15; and SEQ ID NO: 16, SEQ ID NO: 17, and SEQ ID NO: 19. In certain embodiments, the kit further comprises primers and probes for detecting human RNase. In certain embodiments, the kit comprises primers and probe comprising SEQ ID NOs: 19-21.

In certain embodiments, the kits comprise an oligonucleotide useful as a nucleic acid probe, wherein one or more detectable moieties is attached to the nucleic acid probe. In certain embodiments, the one or more detectable moieties is a fluorescent moiety. In certain embodiments, the fluorescent moiety can be selected from the group consisting of fluorescein-family dyes, polyhalofluorescein-family dyes, hexachlorofluorescein-family dyes, coumarin-family dyes, rhodamine-family dyes, cyanine-family dyes, oxazine-family dyes, thiazine-family dyes, squaraine-family dyes, chelated lanthanide-family dyes, and BODIPY®-family dyes.

In certain embodiments, the kits comprise an oligonucleotide useful as a nucleic acid probe, wherein at least one quencher moiety is attached to the nucleic acid probe. In certain embodiments, the quencher moiety can be selected from the group consisting of fluorescein-family dyes, polyhalofluorescein-family dyes, hexachlorofluorescein-family dyes, coumarin-family dyes, rhodamine-family dyes, cyanine-family dyes, oxazine-family dyes, thiazine-family dyes, squaraine-family dyes, chelated lanthanide-family dyes, BODIPY®-family dyes, and non-fluorescent quencher moieties. In certain embodiments, the non-fluorescent quencher moieties can be BHQT™-family dyes, Iowa Black™, or Dabcyl. In other embodiments, the probe comprises at least one detectable moiety, e.g. a fluorescent moiety and at least one quencher moiety. In one embodiment, the probes are labeled using the dual labeled BHQ®.

In certain embodiments, the kits comprise a thermostable DNA polymerase. In certain embodiments, the thermostable DNA polymerase has reverse transcription activity. In certain embodiments, the kits additionally comprise instructions for detecting a nucleic acid of SARS-CoV-2 virus and/or influenza A and/or B viruses and/or the SARS-CoV-2 virus and/or influenza A and/or B viruses, according to the methods and using the compositions disclosed herein. In certain embodiments, the kits include controls including but not limited to positive controls for all of the viruses and human nucleic acid, and negative controls.

### BRIEF DESCRIPTION OF THE FIGURES

FIG. 1 is a schematic of sample addition in performing the Triplex CII-SARS-CoV-2 rRT-PCR Assay.

FIG. 2 shows the results of the alignment of available full length SARS-CoV-2 genomic sequences and primers and probes for N1 and N2. Figure discloses SEQ ID NOS 1, 22, 22, 23, 23, 22, 22, 22, 22, 22, 22, 22, 24, 24, 24, 24, 24, 22, 22, 24, 24, 2, 25-27, 26, 28, 29, 29, 26, 25, 3, 30, 31, 4, 32, 33, 32, 32, 32, 32, 34, 32, 33, 32, 35, 32, 32, 5, 36, 37, 37, 37, 37, 37, 37, 37-39, 37, 37, 37, 6, and 40, respectively, in order of appearance.

FIG. 3 is a plate map used for the clinical evaluation of Triplex CII-SARS-CoV-2 rRT-PCR Assay.

### DETAILED DESCRIPTION

In accordance with the present disclosure, there may be numerous tools and techniques within the skill of the art, such as those commonly used in molecular immunology, cellular immunology, pharmacology, and microbiology. See, e.g., Sambrook et al. (2001) Molecular Cloning: A Laboratory Manual, 3rd ed. Cold Spring Harbor Laboratory Press: Cold Spring Harbor, N.Y.; Ausubel et al. eds. (2005) Current Protocols in Molecular Biology, John Wiley and Sons, Inc.: Hoboken, N.J.; Bonifacino et al. eds. (2005) Current Protocols in Cell Biology, John Wiley and Sons, Inc.: Hoboken, N.J.; Coligan et al. eds. (2005) Current Protocols in Immunology, John Wiley and Sons, Inc.: Hoboken, N.J.; Coico et al. eds. (2005) Current Protocols in Microbiology, John Wiley and Sons, Inc.: Hoboken, N.J.; Coligan et al. eds. (2005) Current Protocols in Protein Science, John Wiley and Sons, Inc.: Hoboken, N.J.; and Enna et al. eds. (2005) Current Protocols in Pharmacology, John Wiley and Sons, Inc.: Hoboken, N.J.

### Definitions

An “amplification reaction” refers to any reaction (e.g., chemical, enzymatic, or other type of reaction) which results in increased copies of a template nucleic acid sequence or increased signal indicating the presence of the template Amplification reactions include, but are not limited to, the polymerase chain reaction (PCR) and ligase chain reaction (LCR) (see U.S. Pat. Nos. 4,683,195 and 4,683,202; PCR Protocols: A Guide to Methods and Applications (Innis et al., eds, 1990)), strand displacement amplification (SDA) (Walker, et al., Nucleic Acids Res. 20(7):1691-6 (1992); Walker PCR Methods Appl 3(1):1-6 (1993)), transcription-mediated amplification (Phyffer, et al., J. Clin. Microbiol. 34:834-841 (1996); Vuorinen, et al., J. Clin. Microbiol. 33:1856-1859 (1995)), nucleic acid sequence-based amplification (NASBA) (Compton, Nature 350(6313):91-2 (1991), rolling circle amplification (RCA) (Lisby, Mol. Biotechnol. 12(1):75-99 (1999)); Hatch, et al., Genet. Anal. 15(2):35-40 (1999)) branched DNA signal amplification (bDNA) (Iqbal, et al., Mol. Cell.

Probes 13(4):315-320 (1999)) and Q-Beta Replicase (Lizardi, et al., Bio/Technology 6:1197 (1988)).

As used herein, a “sample” refers to any substance containing or presumed to contain nucleic acid. The sample can be of natural or synthetic origin and can be obtained by any means known to those of skill in the art. The sample can be a sample of tissue or fluid isolated from an individual or individuals, including, but not limited to, for example, skin, plasma, serum, whole blood, spinal fluid, semen, seminal fluid, lymph fluid, synovial fluid, urine, tears, blood cells, organs, tumors, bronchio-alveolar lavage, nasal swab, nasopharyngeal aspirate, oropharyngeal aspirate, feces, and saliva and also to samples of in vitro cell culture constituents (including but not limited to conditioned medium resulting from the growth of cells in cell culture medium, recombinant cells and cell components). A nucleic acid can be obtained from a biological sample by any procedure known in the art.

As used herein, the term “subject” means any organism including, without limitation, a mammal such as a mouse, a rat, a dog, a guinea pig, a ferret, a rabbit and a primate. In the preferred embodiment, the subject is a human being, a pet or livestock animal.

The term “patient” as used in this application means a human subject.

The terms “detection”, “detect”, “detecting” and the like as used herein means to discover the presence or existence of.

The terms “differentiate”, “differential”, and the like as used herein means to identify or recognize as different.

As used herein, the terms “nucleic acid,” “polynucleotide” and “oligonucleotide” refer to primers, probes, oligomer fragments to be detected, oligomer controls and unlabeled blocking oligomers and is generic to linear polymers of polydeoxyribonucleotides (containing 2-deoxy-D-ribose), polyribonucleotides (containing D-ribose), and any other N-glycoside of a purine or pyrimidine base, or modified purine or pyrimidine bases.

A nucleic acid, polynucleotide or oligonucleotide can comprise phosphodiester linkages or modified linkages including, but not limited to phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages.

A nucleic acid, polynucleotide or oligonucleotide can comprise the five biologically occurring bases (adenine, guanine, thymine, cytosine and uracil) and/or bases other than the five biologically occurring bases. These bases may serve a number of purposes, e.g., to stabilize or destabilize hybridization; to promote or inhibit probe degradation; or as attachment points for detectable moieties or quencher moieties. For example, a polynucleotide can contain one or more modified, non-standard, or derivatized base moieties, including, but not limited to, N6-methyl-adenine, N6-tert-butyl-benzyl-adenine, imidazole, substituted imidazoles, 5-fluorouracil, 5-bromouracil, 5-chlorouracil, 5-iodouracil, hypoxanthine, xanthine, 4-acetylcytosine, 5-(carboxyhydroxymethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acidmethylester, 3-(3-amino-3-N-2-carboxypropyl) uracil, (acp3)w, 2,6-diaminopurine, and 5-propynyl pyrimidine. Other examples of modified, non-standard, or derivatized base moieties may be found in U.S. Pat. Nos. 6,001,611, 5,955,589, 5,844,106, 5,789,562, 5,750,343, 5,728,525, and 5,679,785.

Furthermore, a nucleic acid, polynucleotide or oligonucleotide can comprise one or more modified sugar moieties including, but not limited to, arabinose, 2-fluoroarabinose, xylulose, and a hexose.

It is not intended that the present disclosure be limited by the source of a nucleic acid, polynucleotide or oligonucleotide. A nucleic acid, polynucleotide or oligonucleotide can be from a human or non-human mammal, or any other organism, or derived from any recombinant source, synthesized in vitro or by chemical synthesis. A nucleic acid, nucleotide, polynucleotide or oligonucleotide may be DNA, RNA, cDNA, DNA-RNA, locked nucleic acid (LNA), peptide nucleic acid (PNA), a hybrid or any mixture of the same, and may exist in a double-stranded, single-stranded or partially double-stranded form. A nucleic acid may also be a derivative nucleic acid as described in U.S. Pat. No. 5,696,248. The nucleic acids disclosed herein include both nucleic acids and fragments thereof, in purified or unpurified forms, including genes, chromosomes, plasmids, the genomes of biological material such as microorganisms, e.g., bacteria, yeasts, viruses, viroids, molds, fungi, plants, animals, humans, and the like.

There is no intended distinction in length between the terms nucleic acid, polynucleotide and oligonucleotide, and these terms will be used interchangeably. These terms include double- and single-stranded DNA, as well as double- and single-stranded RNA. Oligonucleotides disclosed herein may be used as primers and/or probes. Thus, oligonucleotides referred to herein as “primers” may act as probes and oligonucleotides referred to as “probes” may act as primer in some embodiments.

The term “residue” as used herein refers to a nucleotide or base within a nucleic acid as defined above. A residue can be any nucleotide known to one of skill in the art without limitation, including all of the biologically occurring nucleotides and non-biologically occurring nucleotides described above.

The term “primer” refers to an oligonucleotide which is capable of acting as a point of initiation of polynucleotide synthesis along a template nucleic acid strand when placed under conditions that permit synthesis of a primer extension product that is complementary to the template strand. The primer can be obtained from a recombinant source, as in a purified restriction fragment, or produced synthetically. Primer extension conditions typically include the presence of four different deoxyribonucleoside triphosphates and an agent with polymerization activity such as DNA polymerase or reverse transcriptase, in a suitable buffer (a “buffer” can include substituents which are cofactors, or which affect pH and/or ionic strength), and at a suitable temperature. The primer is preferably single-stranded for maximum efficiency in amplification. Primers disclosed herein may be between 5 to 500 nucleotides, and in some embodiments will have at least 10, 20, 30, 25, 30, 40, 50, 75, or 100 nucleotides and/or have fewer than 500, 300, 200, 100, 90, 80, 70, 60, 50, 40, 30, 25, or 20 nucleotides.

The term “hybridize” refers to binding of a single-stranded nucleic acid or a locally single-stranded region of a double-stranded nucleic acid to another single-stranded nucleic acid or a locally single-stranded region of a double-stranded nucleic acid having a complementary sequence. As one of skill in the art is aware, it is not necessary for two nucleic acid strands to be entirely complementary to hybridize to each other. Depending on the hybridization conditions, a nucleic acid can hybridize to its complement even if there are few, some, or many mismatches, deletions, or additions in one or both strands. In certain embodiments, the primers and probes of the invention can hybridize to an at least partially complementary nucleic acid selectively, as defined below. In certain embodiments, the primers and probes disclosed herein can hybridize to an at least partially complementary sequence under stringent conditions.

As used herein, the term “probe” refers to an oligonucleotide which can form a duplex structure with a region of a nucleic acid, due to complementarity of at least one sequence in the probe with a sequence in the region. The probe, preferably, does not contain a sequence complementary to sequence(s) of a primer. As discussed below, the probe can be labeled or unlabeled. The 3′ terminus of the probe can be “blocked” to prohibit incorporation of the probe into a primer extension product. “Blocking” can be achieved by using non-complementary bases or by adding a chemical moiety such as biotin or a phosphate group to the 3′ hydroxyl of the last nucleotide, which may, depending upon the selected moiety, serve a dual purpose by also acting as a label for subsequent detection or capture of the nucleic acid attached to the label. Blocking can also be achieved by removing the 3′ hydroxyl or by using a nucleotide that lacks a 3′ hydroxyl such as a dideoxynucleotide.

The term “detectable moiety” as used herein refers to any atom or molecule which can be used to provide a detectable (optionally quantifiable) signal, and which can be attached to a nucleic acid or protein. Detectable moieties may provide signals detectable by fluorescence, radioactivity, colorimetry, gravimetry, X-ray diffraction or absorption, magnetism, enzymatic activity, and the like. Convenient detectable moieties for the present invention include those that facilitate detection of the size of an oligonucleotide fragment.

The term “fluorescent moiety” as used herein refers to a chemical moiety that can emit light under conditions appropriate for the particular moiety. Typically, a particular fluorescent moiety can emit light of a particular wavelength following absorbance of light of shorter wavelength. The wavelength of the light emitted by a particular fluorescent moiety is characteristic of that moiety. Thus, a particular fluorescent moiety can be detected by detecting light of an appropriate wavelength following excitation of the fluorescent moiety with light of shorter wavelength. Examples of fluorescent moieties that can be used in the methods and compositions disclosed herein include, but are not limited to, fluorescein-family dyes, polyhalofluorescein-family dyes, hexachlorofluorescein-family dyes, coumarin-family dyes, rhodamine-family dyes, cyanine-family dyes, oxazine-family dyes, thiazine-family dyes, squaraine-family dyes, chelated lanthanide-family dyes, and BODIPY®-family dyes.

The term “quencher moiety” as used herein refers to a chemical moiety that can absorb energy emitted by a fluorescent moiety when the quencher moiety is sufficiently close to the fluorescent moiety, for example, when both the quencher and fluorescent moiety are linked to a common polynucleotide. This phenomenon is generally known in the art as fluorescent resonance energy transfer (“FRET”). A quencher moiety can re-emit the energy absorbed from a fluorescent moiety in a signal characteristic for that quencher moiety, and thus a quencher can also be a “fluorescent moiety.” Alternatively, a quencher moiety may dissipate the energy absorbed from a fluorescent moiety as heat.

The term “about” or “approximately” means within an acceptable error range for the particular value as determined by one of ordinary skill in the art, which will depend in part on how the value is measured or determined, i.e., the limitations of the measurement system, i.e., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

Unless otherwise explained, all technical and scientific terms used herein have the same meaning as commonly understood by one of ordinary skill in the art to which this disclosure belongs.

### Synthetic Primers and Probes for Detection of SARS-CoV-2 and Influenza

The current disclosure provides for isolated nucleic acid sequences such as primers and probes from specific portions of the particular viral genomes including the nucleoprotein (N) gene, the ORF 1Ab and 3′UTR of SARS-CoV-2, and the matrix gene of influenza A and influenza B. These specific primers and probes were designed considering the possible cross-reactivity based upon sequence alignments and assay sensitivity, thus, the primers and probes disclosed herein are particularly useful in that they can be used in one single sample and/or reaction to detect three different viruses, SARS-CoV-2, influenza A and influenza B, as well as differentiate the viruses in one single sample.

Additionally, the primers and probes of the current disclosure are non-naturally occurring compositions. SARS-CoV-2 and influenza are enveloped, single-stranded RNA viruses. As such, the primers and probes of the current disclosure comprise cDNA that do not occur in nature and the nucleic acid sequences of the current invention are markedly different in structure from naturally occurring viral RNA sequences.

In one aspect, the disclosure provides for at least one primer that is useful in detecting the presence of a nucleic acid of SARS-CoV-2 and/or the SARS-CoV-2 virus itself In certain embodiments, the primers target a 130 nt region located towards the 3′ terminus of the SARS-CoV-2 nucleocapsid (N) gene. In certain embodiments, the primer comprises the nucleotide sequence of SEQ ID NO: 1 (GACCAGGAACTAATCAGACAAGG) or SEQ ID NO: 2 (TCAACCACGTTCCCGAAGG). In certain embodiments, the disclosure is directed to a primer set comprising the primers comprising the nucleotide sequence of SEQ ID NO: 1 and SEQ ID NO: 2.

In certain aspects, the disclosure is directed to oligonucleotide probes comprising isolated nucleic acids as described herein, which probes are suitable for hybridization under suitable conditions to nucleic acids from SARS-CoV-2 virus. In certain embodiments, the probe also detects the 130 nt region located towards the 3′ terminus of the SARS-CoV-2 nucleocapsid (N) gene. In certain embodiments, the probe comprises the nucleic acid of SEQ ID NO: 3 (CGACATTCCGAAGAACGCTGAAGCG).

In a further aspect, the disclosure provides for at least one primer that is useful in detecting the presence of a nucleic acid of SARS-CoV-2 and/or the SARS-CoV-2 virus itself. In certain embodiments, the primers target a different 100 nt region located towards the 3′ terminus of the SARS-CoV-2 nucleocapsid (N) gene than SEQ ID NOs: 1-3. In certain embodiments, the primer comprises the nucleotide sequence of SEQ ID NO: 4 (GCCATCAAATTGGATGACAAAGATC) or SEQ ID NO: 5 (TAGGCTCTGTTGGTGGGAATG). In certain embodiments, the disclosure is directed to a primer set comprising the primers comprising the nucleotide sequence of SEQ ID NO: 4 and SEQ ID NO: 5.

In certain aspects, the disclosure is directed to oligonucleotide probes comprising isolated nucleic acids as described herein, which probes are suitable for hybridization under suitable conditions to nucleic acids from SARS-CoV-2 virus. In certain embodiments, the probe also detects the 100 nt region located towards the 3′ terminus of the SARS-CoV-2 nucleocapsid (N) gene. In certain embodiments, the probe comprises the nucleic acid of SEQ ID NO: 6 (CATTTTGCTGAATAAGCATATTGACGC).

In a further aspect, the disclosure provides for at least one primer that is useful in detecting the presence of a nucleic acid of SARS-CoV-2 from two other unique regions of SARS-CoV-2: the 3′UTR; and Orflab gene.

In certain embodiments, the primer detects the 3′UTR of SARS-CoV-2 and comprises the nucleotide sequence of SEQ ID NO: 7 (AATCARTGTGTAACATTAGGGA) or SEQ ID NO: 8 (AGGCWGCTCTCCCTARCATT). In certain embodiments, the disclosure is directed to a primer set comprising the primers comprising the nucleotide sequence of SEQ ID NO: 7 and SEQ ID NO: 8.

In certain aspects, the disclosure is directed to oligonucleotide probes comprising isolated nucleic acids as described herein, which probes are suitable for hybridization under suitable conditions to nucleic acids from SARS-CoV-2 virus from the 3′UTR of SARS-CoV-2. In certain embodiments, the probe comprises the nucleic acid of SEQ ID NO: 9 (CGCGGAGTACGATCGAGKGTA).

In certain embodiments, the primer detects the ORF1 Ab of SARS-CoV-2 and comprises the nucleotide sequence of SEQ ID NO: 10 (AAGTATTRAGTGARATGGTCATGT) or SEQ ID NO: 11 (GGCAATYTTRTTACCATCAGTWGA). In certain embodiments, the disclosure is directed to a primer set comprising the primers comprising the nucleotide sequence of SEQ ID NO: 10 and SEQ ID NO: 11.

In certain aspects, the disclosure is directed to oligonucleotide probes comprising isolated nucleic acids as described herein, which probes are suitable for hybridization under suitable conditions to nucleic acids from SARS-CoV-2 virus from Orflab gene. In certain embodiments, the probe comprises the nucleic acid of SEQ ID NO: 12 (GATGCCACAACTGCTTATGCTAATAG).

In yet another further aspect, the disclosure provides for at least one primer that is useful in detecting the presence of a nucleic acid of influenza A or influenza A virus itself In certain embodiments, the primers target the matrix gene of influenza A. In certain embodiments, the primer comprises the nucleotide sequence of SEQ ID NO: 13 (CCCCTCAAAGCCGAGATCG) or SEQ ID NO: 14 (GGCACGGTGAGCGTGAA). In certain embodiments, the disclosure is directed to a primer set comprising the primers comprising the nucleotide sequence of SEQ ID NO: 13 and SEQ ID NO: 14.

In certain aspects, the disclosure is directed to oligonucleotide probes comprising isolated nucleic acids as described herein, which probes are suitable for hybridization under suitable conditions to nucleic acids from influenza A. In certain embodiments, the probe comprises the nucleic acid of SEQ ID NO: 15 (ATGGCTAAAGACAAGACCAAT).

In one aspect, the disclosure provides for at least one primer that is useful in detecting the presence of a nucleic acid of influenza B and/or the influenza B virus itself In certain embodiments, the primers target the matrix gene of influenza B. In certain embodiments, the primer comprises the nucleotide sequence of SEQ ID NO: 16 (AAGGCAAAGCAGAACTAGCAGA) or SEQ ID NO: 17 (CAGATAGAGGCACCAATTAGTGCT). In certain embodiments, the disclosure is directed to a primer set comprising the primers comprising the nucleotide sequence of SEQ ID NO: 16 and SEQ ID NO: 17.

In certain aspects, the disclosure is directed to oligonucleotide probes comprising isolated nucleic acids as described herein, which probes are suitable for hybridization under suitable conditions to nucleic acids from influenza B. In certain embodiments, the probe comprises the nucleic acid of SEQ ID NO: 18 (ACACTGTTGGTTYGGTGGGA).

The nucleic acid primers and probes disclosed herein can be prepared by any method known to one of skill in the art without limitation.

One of skill in the art would understand that some bases can be deleted from or added to the end of SEQ ID NOs: 1, 2, 4, 5, 7, 8, 10, 11, 13, 14, 16, and 17, and said primers can still amplify the nucleic acid. Accordingly, this disclosure includes primers wherein some bases are deleted or added to sequences of SEQ ID NOs: 1, 2, 4, 5, 7, 8, 10, 11, 13, 14, 16, and 17.

In addition to the probe nucleotide sequence, the probe can comprise additional nucleotide sequences or other moieties that do not inhibit the methods of the instant disclosure. In convenient embodiments of the disclosure, the probe can comprise additional nucleotide sequences or other moieties that facilitate the methods of the instant disclosure.

For instance, the probe can be blocked at its 3′ terminus to prevent undesired nucleic acid polymerization priming by the probe. Also, moieties may be present within the probe that stabilize or destabilize hybridization of the probe or probe fragments with the nucleotide sequence. The probes of the disclosure can also comprise modified, non-standard, or derivatized nucleotides as defined above.

In certain embodiments of the disclosure, the probe can comprise a detectable moiety. The detectable moiety can be any detectable moiety known by one of skill in the art without limitation. Further, the detectable moiety can be detectable by any means known to one of skill in the art without limitation. For example, the detectable moiety can be detectable by spectroscopic, photochemical, biochemical, immunochemical, or chemical means.

A variety of detectable moieties that can be used to detect the probes of the disclosure, as well as methods for their linkage to the probe, are known to the art and include, but are not limited to, enzymes (e.g., alkaline phosphatase and horseradish peroxidase) and enzyme substrates, radioactive moieties, fluorescent moieties, chromophores, chemiluminescent labels, electrochemiluminescent labels, such as Origin™ (Igen, Rockville, Md.), ligands having specific binding partners, or any other labels that may interact with each other to enhance, alter, or diminish a signal. Should a 5′ nuclease reaction be performed using a thermostable DNA polymerase at elevated temperatures, the detectable moiety should not be degraded or otherwise rendered undetectable by such elevated temperatures.

In certain embodiments, the detectable moiety can be a fluorescent moiety. The fluorescent moiety can be any fluorescent moiety known to one of skill in the art without limitation. In general, fluorescent moieties with wide Stokes shifts are preferred, allowing the use of fluorometers with filters rather than monochromometers and increasing the efficiency of detection. In certain embodiments, the fluorescent moiety can be selected from the group consisting of fluorescein-family dyes (Integrated DNA Technologies, Inc., Coralville, Iowa), polyhalofluorescein-family dyes, hexachlorofluorescein-family dyes, coumarin-family dyes (Molecular Probes, Inc., Eugene, Or), rhodamine-family dyes (Integrated DNA Technologies, Inc.), cyanine-family dyes, oxazine-family dyes, thiazine-family dyes, squaraine-family dyes, chelated lanthanide-family dyes, BODIPY®-family dyes (Molecular Probes, Inc.), and 6-carboxyfluorescein (FAM™) (Integrated DNA Technologies, Inc.). Other examples of fluorescent moieties that can be used in the probes, methods, and kits of the disclosure can be found in U.S. Pat. Nos. 6,406,297, 6,221,604, 5,994,063, 5,808,044, 5,880,287, 5,556,959, and 5,135,717.

In other embodiments, the detectable moiety can be a detectable moiety other than a fluorescent moiety. Among radioactive moieties, ³²P-labeled compounds are preferred. Any method known to one of skill in the art without limitation may be used to introduce ³²P into a probe. For example, a probe may be labeled with ³²P by 5′ labeling with a kinase or by random insertion by nick translation. Detectable moieties that are enzymes can typically be detected by their activity. For example, alkaline phosphatase can be detected by measuring fluorescence produced by action of the enzyme on appropriate substrate compounds. Where a member of specific binding partners is used as detectable moieties, the presence of the probe can be detected by detecting the specific binding of a molecule to the member of the specific binding partner. For example, an antigen can be linked to the probe, and a monoclonal antibody specific for that antigen can be used to detect the presence of the antigen and therefore the probe. Other specific binding partners that can be used as detectable moieties include biotin and avidin or streptavidin, IgG and protein A, and numerous other receptor-ligand couples well-known to the art. Still other examples of detectable moieties that are not fluorescent moieties can be found in U.S. Pat. Nos. 5,525,465, 5,464,746, 5,424,414, and 4,948,882.

The above description of detectable moieties is not meant to categorize the various labels into distinct classes, as the same label may serve in several different modes. For example, ¹²⁵I may serve as a radioactive moiety or as an electron-dense reagent. Horseradish peroxidase may serve as enzyme or as antigen for a monoclonal antibody. Further, one may combine various detectable moieties for desired effect. For example, one might label a probe with biotin, and detect its presence with avidin labeled with ¹²⁵I, or with an anti-biotin monoclonal antibody labeled with horseradish peroxidase. Other permutations and possibilities will be readily apparent to those of ordinary skill in the art and are considered as equivalents within the scope of the instant disclosure.

The method of linking or conjugating the detectable moiety to the probe depends, of course, on the type of detectable moiety or moieties used and the position of the detectable moiety on the probe.

The detectable moiety may be attached to the probe directly or indirectly by a variety of techniques. Depending on the precise type of detectable moiety used, the detectable moiety can be located at the 5′ or 3′ end of the probe, located internally in the probe's nucleotide sequence, or attached to spacer arms of various sizes and compositions to facilitate signal interactions. Using commercially available phosphoramidite reagents, one can produce oligonucleotides containing functional groups (e.g., thiols or primary amines) at either terminus via an appropriately protected phosphoramidite and can attach a detectable moiety thereto using protocols described in, for example, PCR Protocols: A Guide to Methods and Applications, ed. by Innis et al., Academic Press, Inc., 1990.

In certain embodiments, the detectable moiety can be attached to the 5′ end of the probe. In certain embodiments, the detectable moiety can be attached to the 3′ end of the probe. In other embodiments, the detectable moiety can be attached to the probe at a residue that is within the probe. The detectable moiety can be attached to any portion of a residue of the probe. For example, the detectable moiety can be attached to a sugar, phosphate, or base moiety of a nucleotide in the probe. In other embodiments, the detectable moiety can be attached between two residues of the probe.

In certain embodiments, the probe can comprise a fluorescent moiety and a quencher moiety. In such embodiments, the fluorescent moiety can be any fluorescent moiety known to one of skill in the art, as described above. Further, the quencher moiety can be any quencher moiety known to one of skill in the art without limitation. In certain embodiments, the quencher moiety can be selected from the group consisting of fluorescein-family dyes, polyhalofluorescein-family dyes, hexachlorofluorescein-family dyes, coumarin-family dyes, rhodamine-family dyes, cyanine-family dyes, oxazine-family dyes, thiazine-family dyes, squaraine-family dyes, chelated lanthanide-family dyes, BODIPY®-family dyes, and non-fluorescent quencher moieties. In certain embodiments, the non-fluorescent quencher moieties can be BHQT™-family dyes (including the quenchers described in WO 01/86001), Iowa Black™. or Dabcyl (Integrated DNA Technologies, Inc.). Other examples of specific quencher moieties include, for example, but not by way of limitation, TAMRA (N,N,N′,N′-tetramethyl-6-carboxyrhodamine) (Molecular Probes, Inc.), DABCYL (4-(4′-dimethylaminophenylazo)benzoic acid), Iowa Black™. (Integrated DNA Technologies, Inc.), Cy3™ (Integrated DNA Technologies, Inc.) or Cy5™ (Integrated DNA Technologies, Inc.). Other examples of quencher moieties that can be used in the probes, methods, and kits of the disclosure can be found in U.S. Pat. Nos. 6,399,392, 6,348,596, 6,080,068, and 5,707,813.

In certain embodiments, the quencher moiety can be attached to the 5′ end of the probe. In certain embodiments, the quencher moiety can be attached to the 3′ end of the probe. In other embodiments, the quencher moiety can be attached to the probe at a residue that is within the probe. The quencher moiety can be attached to any portion of a residue of the probe. For example, the quencher moiety can be attached to a sugar, phosphate, or base moiety of a nucleotide in the probe. In other embodiments, the quencher moiety can be attached between two residues of the probe.

Exemplary combinations of fluorescent moieties and quencher moieties that can be used in this aspect of the invention include but are not limited dual-labeled BHQ® Probes. Dual-labeled BHQ probes are linear, dual labeled 5′-3′ exonuclease probes incorporating a fluorophore and quencher covalently attached to the 5′ and 3′ ends of the oligonucleotide, respectively. Fluorescence signal is generated through the 5′ exonuclease activity of Taq polymerase, which cleaves off the fluorescent dye-labeled nucleotide from the probe during digestion of the probe hybridized to its complementary sequence in the target strand and thus separating quencher from fluorophore.

In particular, one exemplified probe for the detection of the N gene of SARS-CoV-2 virus, SEQ ID NO: 3, and the exemplified probe for the detection of the 3′UTR of SARS-CoV-2 virus, SEQ ID NO: 9, are modified at the 5′ end with FAM and the 3′ end with BHQ1.

A further exemplified probe for the detection of the N gene of SARS-CoV-2 virus, SEQ ID NO: 6, is modified at the 5′ end with Quasar 670 and the 3′ end with BHQ-3.

The exemplified probe for the detection of the ORF1-Ab of SARS-CoV-2 virus, SEQ ID NO: 12, is modified at the 5′ end with CAL Fluor Red 610 and the 3′ end with BHQ-2.

The exemplified probe for the detection of the influenza A virus, SEQ ID NO: 15, is modified at the 5′ end with CAL Fluor Orange 560 and the 3′ end with BHQ-1 plus.

The exemplified probe for the detection of the influenza B virus, SEQ ID NO: 18, is modified at the 5′ end with CAL Fluor Red 610 and the 3′ end with BHQ-2 plus.

The exemplified probe for human RNase, SEQ ID NO. 21 can be modified at the 5′ end with Quasar 670 and the 3′ end with BHQ-3 or modified at the 5′ end with CAL Fluor Orange 560 and the 3′ end with BHQ-1.

See Table 1.

### Methods of Detection

The present disclosure provides methods for using nucleic acid primers and probes to detect a nucleic acid of certain viruses and/or the virus itself. In some aspects, the present disclosure provides methods for using nucleic acid primers and probes to quantify a nucleic acid of certain viruses in a sample. Any method for using nucleic acid primers and probes to detect a nucleic acid known to one of skill in the art or later developed without limitation can be used to detect a nucleic acid of a detectable virus, as described herein. In certain embodiments, the methods provide using a primer and a probe to detect a nucleic acid of a virus. In other embodiments, the methods provide using more than one primer and a probe to detect a nucleic acid of a virus. In some embodiments, the nucleic acid of one virus is detected in a single sample. In some embodiments, the nucleic acid of more than one virus is detected in a single sample.

One method of detecting a nucleic acid of virus generally comprises contacting a primer hybridized to a nucleic acid of the virus with an enzyme with 5′ nuclease activity. The enzyme with 5′ nuclease activity then fragments a probe hybridized to the nucleic acid of the virus in a 5′ nuclease reaction. The probe can be labeled with a detectable moiety that enables detection of fragmentation of the probe. Such methods are based on those described in U.S. Pat. Nos. 6,214,979, 5,804,375, 5,487,972 and 5,210,015.

In a 5′ nuclease reaction, the nucleic acid, primer and probe can be contacted with any enzyme known by one of skill in the art to have 5′ to 3′ nuclease activity without limitation. The conditions are preferably chosen to permit the polymerase to cleave the probe and release a plurality of fragments of the probe from the nucleic acid. Preferred enzymes with 5′ nuclease activity include template-dependent nucleic acid polymerases. Known native and recombinant forms of such polymerases include, for example, E. coli DNA polymerase I (Fermentas, Inc., Hanover, Md.), Bacillus stearothermophilus DNA polymerase, and Thermococcus littoralis DNA polymerase.

In some embodiments, the enzymes with 5′ nuclease activity are thermostable and thermoactive nucleic acid polymerases. Such thermostable polymerases include, but are not limited to, native and recombinant forms of polymerases from a variety of species of the eubacterial genera Thermus, Thermatoga, and Thermosipho.

A 5′ nuclease reaction comprises contacting the nucleic acid to be detected with a primer, a probe, and an enzyme having 5′ to 3′ nuclease activity, under conditions in which the primer and the probe hybridize to the nucleic acid. Components of a 5′ nuclease reaction can contact the nucleic acid to be detected in any order, e.g., the primer can contact the nucleic acid to be detected first, followed by the probe and enzyme with 5′ nuclease activity, or alternatively the enzyme with 5′ nuclease activity can contact the nucleic acid to be detected first, followed by the probe and primer. In certain embodiments, more than one primer or probe may be added to a 5′ nuclease reaction. In certain preferred embodiments, a pair of primers can contact the nucleic acid in a 5′ nuclease reaction. The primer can be any primer capable of priming a DNA synthesis reaction. Where only one primer is used, the primer should hybridize to the nucleic acid upstream of the probe, i.e., the 3′ end of the primer should point toward the 5′ end of the probe. The 3′ end of the primer can hybridize adjacent to the 5′ end of the probe, or the 3′ end of the primer can hybridize further upstream of the 5′ end of the probe. Where more than one primer is used, at least one primer should hybridize to the nucleic acid to be detected upstream of the probe, as described above.

Certain embodiments of the 5′ nuclease reactions of the present invention are based on several 5′ nuclease reactions that are known to those of skill in the art. Examples of such reactions are described in detail, for instance, in U.S. Pat. No. 5,210,015.

Briefly, in a 5′ nuclease reaction, a target nucleic acid is contacted with a primer and a probe under conditions in which the primer and probe hybridize to a strand of the nucleic acid. The nucleic acid, primer and probe are also contacted with an enzyme, for example a nucleic acid polymerase, having 5′ to 3′ nuclease activity. Nucleic acid polymerases possessing 5′ to 3′ nuclease activity can cleave the probe hybridized to the nucleic acid downstream of the primer. The 3′ end of the primer provides a substrate for extension of a new nucleic acid as based upon the template nucleic acid by the nucleic acid polymerase. As the polymerase extends the new nucleic acid, it encounters the 5′ end of the probe and begins to cleave fragments from the probe.

The primer and probe can be designed such that they hybridize to the target nucleic acid in close proximity to each other such that binding of the nucleic acid polymerase to the 3′ end of the primer puts it in contact with the 5′ end of the probe. In this process, nucleic acid extension is not required to bring the nucleic acid polymerase into position to accomplish the cleavage. The term “polymerization-independent cleavage” refers to this process.

Alternatively, if the primer and probe anneal to more distantly spaced regions of the nucleic acid, nucleic acid extension must occur before the nucleic acid polymerase encounters the 5′ end of the probe. As the polymerization continues, the polymerase progressively cleaves fragments from the 5′ end of the probe. This cleaving continues until the remainder of the probe has been destabilized to the extent that it dissociates from the template molecule. The term “polymerization-dependent cleavage” refers to this process.

In either process, a sample is provided which contains the nucleic acid. If the nucleic acid is double-stranded, it should first be denatured, e.g., the strands of the nucleic acid separated from each other. Any suitable denaturing method, including physical, chemical, or enzymatic means, known to one of skill in the art without limitation can be used to separate the nucleic acid strands.

It should be noted that the viruses that can be detected with the primers, probes, methods, and kits of the disclosure are single-stranded RNA viruses. Accordingly, denaturation of the native viral genome is not required to detect an unamplified viral genome. However, if the native viral genome is reverse-transcribed into DNA according to certain embodiments, denaturation of the amplified viral nucleic acids is necessary prior to detection with the disclosed primers and probes.

If the nucleic acid to be detected is RNA, the RNA can either be used as an RNA template for a 5′ nuclease reaction as described above, or the RNA can be used as a template for reverse-transcription into cDNA, or both simultaneously. In certain embodiments, the RNA can be detected without reverse-transcription into cDNA using the disclosed methods. Polymerization-independent cleavage methods as described above are particularly well-suited for such embodiments. In other embodiments, the RNA can be first reverse-transcribed into cDNA in the absence of a probe, and then the cDNA product can be detected according to the disclosed methods. In still other embodiments, the RNA can be reverse-transcribed in the presence of a probe, simultaneously producing cDNA that can subsequently be amplified and/or detected and detecting the presence of the RNA by assessing fragmentation of the probe as described herein.

Where the RNA is reverse-transcribed in the absence of a probe, the RNA can be reverse transcribed into cDNA by any method known to one of skill in the art. The products of such reverse transcription can then be detected like any detectable nucleic acid according to the methods described herein.

Where the RNA is reverse-transcribed in the presence of a probe, the RNA can be reverse-transcribed by a DNA polymerase with 5′-3′ nuclease activity that can use RNA as a template for DNA strand synthesis. As with all known DNA polymerase synthesis activities, such synthesis requires the presence of a primer, such as those described herein. The DNA polymerase that can use RNA is a template is preferably thermostable, so that multiple cycles of denaturation and DNA synthesis can occur without destroying the polymerase. Further, the DNA polymerase used for reverse transcription can preferably also synthesize DNA using a DNA template. Such polymerases are described in, for example, U.S. Pat. Nos. 6,468,775 (Carboxydothermus hydrogenformans DNA polymerase), 5,968,799 (Thermosipho africanus DNA polymerase), 5,736,373 (Bacillus pallidus DNA polymerase), 5,674,738 (Thermus species Z05 DNA polymerase), and 5,407,800 (Thermus aquaticus and Thermus thermophilus DNA polymerases). In addition, methods and compositions for reverse transcribing an RNA using a thermostable DNA polymerase with reverse transcription activity are described in U.S. Pat. Nos. 5,693,517, 5,561,058, 5,405,774, 5,352,600, 5,310,652, and 5,079,352.

Whether RNA or DNA, the denatured nucleic acid strand is then contacted with a primer and a probe under hybridization conditions, which enable the primer and probe to bind to the nucleic acid strand. In certain embodiments, two primers can be used to amplify the nucleic acid. In such embodiments, the two primers can be selected so that their relative positions along the nucleic acid are such that an extension product synthesized from one primer, after the extension produce is separated from its template (complement), can serve as a template for the extension of the other primer to yield an amplified product of defined length. The length of the product depends on the length of the sequence between the two primers and the length of the two primers themselves.

The probe preferably hybridizes to the nucleic acid to be detected before the polymerase binds the nucleic acid and primer and begins to extend the new nucleic acid strand from the primer based upon the template of the detectable nucleic acid. It is possible for the polymerase to bind the primer and nucleic acid to be detected before the probe contacts the detectable nucleic acid; however, this arrangement can result in decreased probe fragmentation unless multiple cycles of primer extension are performed, as in a preferred PCR based 5′ nuclease reaction as described below. Accordingly, it is preferable that the probe hybridize to the nucleic acid to be detected before primer extension by the polymerase begins.

A variety of techniques known to one of skill in the art can be employed to enhance the likelihood that the probe will hybridize to the detectable nucleic acid before primer extension polymerization reaches this duplex region, or before the polymerase attaches to the upstream oligonucleotide in the polymerization-independent process. For example, short primer molecules generally require cooler temperature to form sufficiently stable hybrid complexes with the nucleic acid. Therefore, the probe can be designed to be longer than the primer so that the probe anneals preferentially to the nucleic acid at higher temperatures relative to primer annealing.

One can also use primers and probes having differential thermal stability based upon their nucleotide composition. For example, the probe can be chosen to have greater G/C content and, consequently, greater thermal stability than the primer. Alternatively or additionally, one or more modified, non-standard or derivatized DNA bases may be incorporated into primers or probes to result in either greater or lesser thermal stability in comparison to primers or probes having only conventional DNA bases. Examples of such modified, non-standard or derivatized bases may be found in U.S. Pat. Nos. 6,320,005, 6,174,998, 6,001,611, and 5,990,303.

Further, the temperature of the reaction can also be varied to take advantage of the differential thermal stability of the probe and primer. For example, following denaturation at high temperatures as described above, the reaction can be incubated at an intermediate temperature which permits probe but not primer binding, followed by a further temperature reduction to permit primer annealing and subsequent extension.

Template-dependent extension of the oligonucleotide primer(s) is catalyzed by a DNA polymerase in the presence of adequate amounts of the four deoxyribonucleoside triphosphates (dATP, dGTP, dCTP, and dTTP) or analogs, e.g., dUTP, as discussed above, in a reaction medium which is comprised of the appropriate salts, metal cations, and pH buffering system. Suitable polymerizing agents are enzymes known to catalyze primer and template-dependent DNA synthesis and possess the 5′ to 3′ nuclease activity. Such enzymes include, for example, Escherichia coli DNA polymerase I, Thermus thermophilus DNA polymerase, Bacillus stearothermophilus DNA polymerase, Thermococcus littoralis DNA polymerase, Thermus aquaticus DNA polymerase, Thermatoga maritima DNA polymerase and Thermatoga neapolitana DNA polymerase and Z05 DNA polymerase. Further, the reaction conditions for performing DNA synthesis using these DNA polymerases are well known in the art. To be useful in the methods of the present invention, the polymerizing agent should possess 5′ nuclease activity that can efficiently cleave the oligonucleotide and release labeled fragments so that a detectable signal is directly or indirectly generated.

The products of the synthesis are duplex molecules consisting of the template strands and the primer extension strands. Byproducts of this synthesis are probe fragments which can consist of a mixture of mono-, di- and oligo-nucleotide fragments. In preferred embodiments, repeated cycles of denaturation, probe and primer annealing, and primer extension and cleavage of the probe can be performed, resulting in exponential accumulation of the amplified region defined by the primers and exponential generation of labeled fragments. Such repeated thermal cycling is generally known in the art as the polymerase chain reaction (PCR). Sufficient cycles can be performed to achieve fragment a sufficient amount of the probe to distinguish positive reactions, i.e., the nucleic acid to be detected is present, from negative reactions, i.e., the nucleic acid to be detected is not present. Generally, positive reactions will exhibit a signal that is several orders of magnitude greater than a negative reaction.

In certain embodiments, the PCR reaction is carried out as an automated process which utilizes a thermostable enzyme. In this process the reaction mixture is cycled through a denaturing step, a probe and primer annealing step, and a synthesis step, whereby cleavage and displacement occur simultaneously with primer dependent template extension. In certain of such embodiments, the nucleic acids to be detected can be amplified in the absence of a detectably-labeled probe, followed by detection of the amplification product in a separate reaction. Alternatively, the nucleic acids to be detected can be amplified in the presence of the probe, allowing amplification and detection in a single reaction.

Temperature stable polymerases are preferred in this automated process because the preferred way of denaturing the double stranded extension products is by exposing them to a high temperature during the PCR cycle. For example, U.S. Pat. No. 4,889,818 discloses a representative thermostable enzyme isolated from Thermus aquaticus. Additional representative temperature stable polymerases include, e.g., polymerases extracted from the thermostable bacteria Thermus flavus, Thermus Tuber, Thermus thermophilus, Bacillus stearothermophilus (which has a somewhat lower temperature optimum than the others listed), Thermus lacteus, Thermus rubens, Thermotoga maritima, Thermococcus littoralis, Methanothermus fervidus, and Pyrococcus furiosus (Stratagene, La Jolla, Calif.). As described above, certain of these thermostable polymerases can synthesize DNA from an RNA template. Where an RNA molecule is to be detected according to the methods of the invention, a DNA polymerase that can synthesize DNA from an RNA template, i.e., with reverse transcription activity, should be used.

The primer and probes described herein can be used in methods and systems utilizing a PCR format including those described above, many of which are commercially available and in an automated system. Exemplified herein is an assay denoted Triplex CII-SARS-CoV-2 rRT-PCR which utilizes SEQ ID NOs: 1-6 as well as controls SEQ ID NOs: 19-21. See Examples 2-7 and Table 2. In the exemplified assay, dual-labeled BHQ probes are used which are linear, dual labeled 5′-3′ exonuclease probes incorporating a fluorophore and quencher covalently attached to the 5′ and 3′ ends of the oligonucleotide, respectively. Fluorescence signal is generated through the 5′ exonuclease activity of Taq polymerase, which cleaves off the fluorescent dye-labeled nucleotide from the probe during digestion of the probe hybridized to its complementary sequence in the target strand and thus separating quencher from fluorophore. The five primer and probe sets were designed to detect RNA from the nucleocapsid gene of SARS-CoV-2 virus in nasal and oral aspirates from patients presenting with signs and symptoms of the respective virus infection and/or epidemiological risk factors consistent with viral exposure. After patient specimen collection and receipt by the laboratory, total nucleic acids can be isolated from samples using the NucliSENS® easyMag® automated extraction platform (bioMérieux). The purified nucleic acids can be reverse transcribed and amplified by using the RNA UltraSense™ One-Step Quantitative RT-PCR System (ThermoFisher) with thermal cycling and detection on the CFX96 Touch™ Real-Time PCR Detection System (Bio-Rad). In the process, the specific probe anneals to a specific target sequence located between the specific forward and reverse primers generating a fluorescent signal, which is measured during the end of the elongation phase of the PCR cycle. With each cycle of PCR, more probes are digested, resulting in an increase in fluorescence that is proportional to the amount of target nucleic acid. Fluorescence signal intensity is analyzed and data collected by the CFX Manage™ Software.

Using the exemplified assay and the primers and probes SEQ ID NOs: 1-6, the sensitivity, specificity, and cross-reactivity of the assay was evaluated and it was determined that the assay performed as required to detect SARS-CoV-2. See Examples 2-6. Additionally, the performance characteristics of the assay using clinical samples known to be positive or negative for particular viruses was also evaluated and found to perform as required. See Example 7.

Also exemplified herein is an assay denoted Quadraplex CII-SARS-CoV-2 rRT-PCR which utilizes SEQ ID NOs: 1-3, and 13-18 as well as controls SEQ ID NOs: 19-21. See Examples 8-11. This Quadraplex CII-SARS-CoV-2 rRT-PCR works the same as the Triplex CII-SARS-CoV-2 rRT-PCR but primers and probes which recognize both SARS-CoV-2 and influenza A and influenza B are used which allows the differential detection of SARS-CoV-2 virus from influenza virus.

The present invention includes methods and systems for the detection of nucleic acid from SARS-CoV-2 and/or influenza in any sample utilizing the primers and probes of the present disclosure.

The methods and systems of the present disclosure may be used to detect nucleic acids from SARS-CoV-2 and/or influenza in research and clinical settings.

A preferred sample is a biological sample. A biological sample may be obtained from a tissue of a subject or bodily fluid from a subject including but not limited to nasopharyngeal aspirate, oropharyngeal aspirate, blood, cerebrospinal fluid, saliva, serum, plasma, urine, sputum, bronchial lavage, pericardial fluid, or peritoneal fluid, or a solid such as feces. Preferred samples include but are not limited to nasal swabs, nasopharyngeal aspirates, oropharyngeal aspirates, feces, and saliva.

The subject may be any animal, particularly a vertebrate and more particularly a mammal, including, without limitation, a cow, dog, human, monkey, mouse, pig, or rat. In one embodiment, the subject is a human.

A sample may also be a research, clinical, or environmental sample. One such sample is waste water.

Additional applications include, without limitation, detection of the screening of blood products (e.g., screening blood products for infectious agents), biodefense, food safety, environmental contamination, forensics, and genetic-comparability studies. The present disclosure also provides methods and systems for detecting viral nucleic acids in cells, cell culture, cell culture medium and other compositions used for the development of pharmaceutical and therapeutic agents.

Thus, one embodiment of the present disclosure is a system for the detection of nucleic acid from SARS-CoV-2 or of detection of the virus itself, in any sample. The system includes at least one subsystem wherein the subsystem includes: the primer groups comprising SEQ ID NOs: 1 and 2; SEQ ID NOs: 4 and 5; SEQ ID NOs: 7 and 8; and SEQ ID NOs: 10 and 11; and probes comprising SEQ ID NOs: 3, 6, 9, and 12. In some embodiments, the system includes at least one subsystem wherein the subsystem includes: the primer groups SEQ ID NOs: 1 and 2; and SEQ ID NOs: 4 and 5; and probes comprising SEQ ID NOs: 3 and 6. The system can also include additional subsystems for the purpose of: extraction of nucleic acids from the sample; reverse transcribing the nucleic acid from the sample; amplifying the reaction; and detection of the amplification products.

A further embodiment of the present disclosure is a system for the detection of nucleic acid from SARS-CoV-2 and/or influenza or of detection of the virus itself, in any sample. The system includes at least one subsystem wherein the subsystem includes: the primer groups comprising SEQ ID NOs: 1 and 2; SEQ ID NOs: 4 and 5; SEQ ID NOs: 7 and 8; SEQ ID NOs: 10 and 11; SEQ ID NOs: 13 and 14; and SEQ ID NOs: 16 and 17, and probes comprising SEQ ID NOs: 3, 6, 9, 12, 15, and 18. In some embodiments, the system includes at least one subsystem wherein the subsystem includes: the primer groups comprising SEQ ID NOs: 1 and 2; SEQ ID NOs: 13 and 14; and SEQ ID NOs: 15 and 16, and probes comprising SEQ ID NOs: 3, 15, and 18. The system can also include additional subsystems for the purpose of: extraction of nucleic acids from the sample; reverse transcribing the nucleic acid from the sample; amplifying the reaction; and detection of the amplification products.

The present disclosure also provides a method for detecting nucleic acid from SARS-CoV-2, or of detection of the virus itself, in any sample, including the steps of: obtaining the sample; extracting nucleic acid from the sample; contacting the nucleic acid in the sample with at least one primer selected from the group consisting of SEQ ID NOs: 1, 2, 4, 5, 7, 8, 10, and 11; subjecting the nucleic acid and primer to amplification conditions; and detecting the presence of amplification product, wherein the presence of the amplification products indicates the presence of nucleic acid of the virus and the virus in the sample.

In a further embodiment, the method comprises contacting the nucleic acid from the sample with primer groups comprising SEQ ID NOs: 1 and 2; SEQ ID NOs: 4 and 5; SEQ ID NOs: 7 and 8; and SEQ ID NOs: 10 and 11. In a further embodiment, the method comprises further contacting the nucleic acid from the sample with probes comprising SEQ ID NOs: 3, 6, 9, and 12. In yet a further embodiment, the probes are detectable in order to detect the presence of the amplification product in the sample.

In a further embodiment, the method comprises contacting the nucleic acid from the sample with primer groups comprising SEQ ID NOs: 1 and 2; and SEQ ID NOs: 4 and 5. In a further embodiment, the method comprises further contacting the nucleic acid from the sample with probes comprising SEQ ID NOs: 3, and 6. In yet a further embodiment, the probes are detectable in order to detect the presence of the amplification product in the sample.

Because these specific primers and probes were designed considering the possible cross-reactivity based upon sequence alignments and assay sensitivity, they are particularly useful in that they can be used in one single sample and/or reaction to detect three different viruses, SARS-CoV-2, influenza A and influenza B, as well as differentiate the viruses in one single sample.

Thus, the present disclosure also provides a method for detecting nucleic acid from SARS-CoV-2 and/or influenza, or of detection of the virus itself, in any sample, including the steps of: obtaining the sample; extracting nucleic acid from the sample; contacting the nucleic acid in the sample with at least one primer selected from the group consisting of SEQ ID NOs: 1, 2, 4, 5, 7, 8, 10, 11, 13, 14, 16, and 17; subjecting the nucleic acid and primer to amplification conditions; and detecting the presence of amplification product, wherein the presence of the amplification products indicates the presence of nucleic acid of the virus and the virus in the sample.

In a further embodiment, the method comprises contacting the nucleic acid from the sample with primer groups comprising SEQ ID NOs: 1 and 2; SEQ ID NOs: 4 and 5; SEQ ID NOs: 7 and 8; SEQ ID NOs: 10 and 11; SEQ ID NOs: 13 and 14; and SEQ ID NOs: 16 and 17. In a further embodiment, the method comprises further contacting the nucleic acid from the sample with probes comprising SEQ ID NOs: 3, 6, 9, 12, 15, and 18. In yet a further embodiment, the probes are detectable in order to detect the presence of the amplification product in the sample.

In a further embodiment, the method comprises contacting the nucleic acid from the sample with primer groups comprising SEQ ID NOs: 1 and 2; SEQ ID NOs: 13 and 14; and SEQ ID NOs: 16 and 17. In a further embodiment, the method comprises further contacting the nucleic acid from the sample with probes comprising SEQ ID NOs: 3, 15, and 18. In yet a further embodiment, the probes are detectable in order to detect the presence of the amplification product in the sample.

The present disclosure also provides a method for detecting and differentiating SARS-CoV-2 from influenza, in any sample, including the steps of: obtaining the sample; extracting nucleic acid from the sample; contacting the nucleic acid in the sample with at least one primer selected from the group consisting of SEQ ID NOs: 1, 2, 4, 5, 7, 8, 10, 11, 13, 15, 16, and 17; subjecting the nucleic acid and primer to amplification conditions; and detecting the presence of amplification product, wherein the presence of the amplification products indicates the presence of nucleic acid of the virus in the sample.

In a further embodiment, the method comprises contacting the nucleic acid from the sample with primer groups comprising SEQ ID NOs: 1 and 2; SEQ ID NOs: 4 and 5; SEQ ID NOs: 7 and 8; SEQ ID NOs: 10 and 11; SEQ ID NOs: 13 and 14; and SEQ ID NOs: 16 and 17. In a further embodiment, the method comprises further contacting the nucleic acid from the sample with probes comprising SEQ ID NOs: 3, 6, 9, 12, 15, and 18. In yet a further embodiment, the probes are detectable in order to detect the presence of the amplification product in the sample.

In a further embodiment, the method comprises contacting the nucleic acid from the sample with primer groups comprising SEQ ID NOs: 1 and 2; SEQ ID NOs: 13 and 14; and SEQ ID NOs: 16 and 17. In a further embodiment, the method comprises further contacting the nucleic acid from the sample with probes comprising SEQ ID NOs: 3, 15, and 18. In yet a further embodiment, the probes are detectable in order to detect the presence of the amplification product in the sample.

### Kits

In another aspect, the present disclosure provides kits that can be used to detect a nucleic acid of a virus or the virus itself. The kit can be used to detect nucleic acid from SARS-CoV-2 and/or influenza viruses and/or the detection of the SARS-CoV-2 and/or influenza virus. The kit also can be used to detect and differentiate SARS-CoV-2 from influenza viruses. In certain embodiments, the kit comprises a probe. In some embodiments, the kit comprises a primer. In some embodiments, the kit comprises a combination of one or more of the primers and probes disclosed herein.

In certain embodiments, the kit comprises a combination of one or more of the primers and probes disclosed herein. In certain embodiments the kit comprises one or more primers chosen from the group consisting of SEQ ID NOs: 1, 2, 4, 5, 7, 8, 10, and 11. In certain embodiments, the kit comprises one or more probes chosen from the group consisting of SEQ ID NOs: 3, 6, 9, and 12.

In certain embodiments the kit comprises one or more primers chosen from the group consisting of SEQ ID NOs: 1, 2, 4, 5, 7, 8, 10, 11, 13, 15, 16, and 17. In certain embodiments, the kit comprises one or more probes chosen from the group consisting of SEQ ID NOs: 3, 6, 9, 12. 15, and 18.

In certain embodiments, the kit further comprises primers and probes for positive control sequences. In certain embodiments, the kit further comprises primers and probes for detecting human RNase. In certain embodiments, the kit comprises primers and probe comprising SEQ ID NOs: 19-21.

In certain embodiments, the kits comprise an oligonucleotide useful as a nucleic acid probe, wherein one or more detectable moieties are attached to the nucleic acid probe. In certain embodiments, the one or more detectable moieties are a fluorescent moiety. In certain embodiments, the fluorescent moiety can be selected from the group consisting of fluorescein-family dyes, polyhalofluorescein-family dyes, hexachlorofluorescein-family dyes, coumarin-family dyes, rhodamine-family dyes, cyanine-family dyes, oxazine-family dyes, thiazine-family dyes, squaraine-family dyes, chelated lanthanide-family dyes, and BODIPY®-family dyes.

In certain embodiments, the kits comprise an oligonucleotide useful as a nucleic acid probe, wherein at least one quencher moiety is attached to the nucleic acid probe. In certain embodiments, the quencher moiety can be selected from the group consisting of fluorescein-family dyes, polyhalofluorescein-family dyes, hexachlorofluorescein-family dyes, coumarin-family dyes, rhodamine-family dyes, cyanine-family dyes, oxazine-family dyes, thiazine-family dyes, squaraine-family dyes, chelated lanthanide-family dyes, BODIPY®-family dyes, and non-fluorescent quencher moieties. In certain embodiments, the non-fluorescent quencher moieties can be BHQT™-family dyes, Iowa Black™, or Dabcyl. In other embodiments, the probe comprises at least one detectable moiety, e.g. a fluorescent moiety and at least one quencher moiety.

In certain embodiments, the kits comprise a thermostable DNA polymerase. In certain embodiments, the thermostable DNA polymerase has reverse transcription activity. In certain embodiments, the kits of the invention additionally comprise instructions for detecting a nucleic acid of SARS-CoV-2 and/or influenza according to the disclosed methods.

In other embodiments, the kits comprise one or more containers to hold the components of the kit.

In certain embodiments, the kits can contain a composition comprising a primer disclosed herein. The kits can also contain a composition comprising a probe disclosed herein. The kits can further contain a composition comprising a thermostable DNA polymerase. In some embodiments, the thermostable DNA polymerase is selected from the group of Carboxydothermus hydrogenformans DNA polymerase, Thermosipho africanus DNA polymerase, Bacillus pallidus DNA polymerase, Thermus species Z05 DNA polymerase, Thermus aquaticus DNA polymerase, Thermus thermophilus DNA polymerase, Thermatoga maritima DNA polymerase, Thermatoga neapolitana DNA polymerase and Thermus sps17 DNA polymerase The compositions comprising a disclosed primer or probe or a thermostable DNA polymerase can further comprise additional reagents. For example, the compositions can comprise suitable preservatives prevent degradation of the composition, suitable buffers to modulate the pH of the composition, suitable diluents to alter the viscosity of the compositions, and the like.

The kits can additionally comprise other reagents for carrying out 5′ nuclease reactions, as described above. In addition, the kits can comprise reagents to facilitate the detection of a fragmented probe that indicates the presence of a nucleic acid of SARS-CoV-2 and/or influenza.

One embodiment of the present disclosure is a kit comprising various containers comprising various components for the detection of SARS-CoV-2 (“Triplex CII-SARS-CoV-2 rRT-PCR”) (see Table 2).

A further embodiment of the present disclosure is a kit comprising containers comprising the various components for the differential detection of SARS-CoV-2 and influenza A and influenza B (“Quadraplex CII-SARS-CoV-2 rRT-PCR”) (see Table 13).

### EXAMPLES

The present invention may be better understood by reference to the following non-limiting examples, which are presented in order to more fully illustrate the preferred embodiments of the invention. They should in no way be construed to limit the broad scope of the invention.

### Example 1—Primers and Probes for Use in the Assays

The following probes and primers were used in the Examples.

TABLE 1
Primer and probe sets for detection of SARS-COV-2,
influenza A, influenza B, and human RNase P RNAs

| Primer/ | | | 5′ | 3′ | |
|---|---|---|---|---|---|
| Probe | Primer/ | Primer/Probe | Modifi- | Modifi- | |
| name | Probe | sequence | cation | cation | Gene |
| nCoV-NF1 | Forward | GACCAGGAACTAATC | | | SARS- |
| | primer | AGACAAGG (SEQ ID | | | CoV-2 |
| | | NO: 1) | | | nucleo- |
| | | | | | capsid |
| nCoV-NR1 | Reverse | TCAACCACGTTCCCGA | | | |
| | primer | AGG (SEQ ID NO: 2) | | | |
| nCoV-NP1 | Probe | CGACATTCCGAAGAAC | FAM | BHQ-1 | |
| | | GCTGAAGCG (SEQ ID | | | |
| | | NO: 3) | | | |
| nCoV-NF2 | Forward | GCCATCAAATTGGATG | | | |
| | primer | ACAAAGATC (SEQ ID | | | |
| | | NO: 4) | | | |
| nCoV-NR2 | Reverse | TAGGCTCTGTTGGTGG | | | SARS- |
| | primer | GAATG (SEQ ID NO: 5) | | | CoV-2 |
| | | | | | nucleo- |
| | | | | | capsid |
| nCoV-NP2 | Probe | CATTTTGCTGAATAAG | Quasar 670 | BHQ-3 Plus | |
| | | CATATTGACGC (SEQ | | | |
| | | ID NO: 6) | | | |
| nCoV-QF1 | Forward | AATCARTGTGTAACAT | | | |
| | primer | TAGGGA (SEQ ID NO: 7) | | | |
| nCoV-QR1 | Reverse | AGGCWGCTCTCCCTAR | | | SARS- |
| | primer | CATT (SEQ ID NO: 8) | | | CoV-2 |
| | | | | | 3′UTR |
| nCoV-QP1 | Probe | CGCGGAGTACGATCG | FAM | BHQ-1 | |
| | | AGKGTA (SEQ ID NO: 9) | | | |
| nCoV-QF2 | Forward | AAGTATTRAGTGARAT | | | |
| | primer | GGTCATGT (SEQ ID | | | |
| | | NO:10) | | | |
| nCoV-QR2 | Reverse | GGCAATYTTRTTACCA | | | ORF1 Ab |
| | primer | TCAGTWGA (SEQ ID | | | |
| | | NO: 11) | | | |
| nCoV-QP2 | Probe | GATGCCACAACTGCTT | CAL Fluor | BHQ-2 | Matrix |
| | | ATGCTAATAG (SEQ ID | Red 610 | | |
| | | NO 12) | | | |
| Influenza-A- | Forward | CCCCTCAAAGCCGAGA | | | |
| MF | Primer | TCG (SEQ ID NO: 13) | | | |
| Influenza-A- | Reverse | GGCACGGTGAGCGTG | | | |
| MR | primer | AA (SEQ ID NO: 14) | | | |
| Influenza-A- | Probe | ATGGCTAAAGACAAG | CAL Fluor | BHQ-1 plus | |
| MP1 | | ACCAAT | Orange 560 | | |
| | | (SEQ ID NO: 15) | | | |
| Influenza-B- | Forward | AAGGCAAAGCAGAAC | | | Matrix |
| MF | primer | TAGCAGA | | | |
| | | (SEQ ID NO: 16) | | | |
| Influenza-B- | Reverse | CAGATAGAGGCACCA | | | |
| MR | primer | ATTAGTGCT | | | |
| | | (SEQ ID NO: 17) | | | |
| Influenza-B- | Probe | ACACTGTTGGTTYGGT | CAL Fluor | BHQ-2 plus | |
| MP1 | | GGGA | Red 610 | | |
| | | (SEQ ID NO: 18) | | | |
| RP-F | Forward | AGATTTGGACCTGCGA | | | Human |
| | primer | GCG | | | RNase P |
| | | (SEQ ID NO: 19) | | | |
| RP-R | Reverse | GAGCGGCTGTCTCCAC | | | |
| | primer | AAGT | | | |
| | | (SEQ ID NO: 20) | | | |
| RP-P | Probe | TTCTGACCTGAAGGCT | Quasar 670 | BHQ-3 | |
| | | CTGCGCG | CAL Fluor | BHQ-1 | |
| | | (SEQ ID NO: 21) | Orange 560 | | |

### Example 2—Reagents and Materials for the Triplex CII-SARS-CoV-2 rRT-PCR

The Triplex CII-SARS-CoV-2 rRT-PCR assay is intended for the qualitative detection of nucleic acid from the SARS-CoV-2 in nasopharyngeal (NPS) and oropharyngeal (OPS) swabs collected from individuals with suspected COVID-19. Results are for the detection and identification of SARS-CoV-2 RNA. The SARS-CoV-2 RNA is generally detectable in respiratory specimens during the acute phase of infection. Positive results are indicative of active infection with SARS-CoV-2. Other samples which can be used in the assay include but are not limited to nasal swabs, feces and saliva.

The Triplex CII-SARS-CoV-2 rRT-PCR assay uses two primer-probe sets to detect the nucleocapsid (N) gene of SARS-CoV-2 (SEQ ID NOs: 1-6) and a primer-probe set targeting the human RNase P housekeeping gene (SEQ ID NOs: 19-21) (Table 1). The Triplex CII-SARS-CoV-2 rRT-PCR assay can detect three targets (two targets in SARS-CoV-2 N gene and human RNAse P) simultaneously.

The Triplex CII-SARS-CoV-2 rRT-PCR assay is a one-step rRT-PCR test. The assay includes primers and dual-labeled probes to be used in the in vitro qualitative detection of isolated RNA from SARS-CoV-2 and the host control transcript RNase P from clinical specimens. The assay is a real-time reverse transcription polymerase chain reaction (rRT-PCR) test. The two SARS-CoV-2 primer and probe sets were designed to detect RNA from SARS-CoV-2 in respiratory specimens from patients as recommended for testing by public health authority guidelines. Dual-labeled probes are linear oligonucleotides, incorporating a fluorophore and quencher covalently attached to the 5′ and 3′ nucleotides of the oligonucleotide, respectively. Fluorescence signal is generated through the 5′ exonuclease activity of Taq polymerase, which cleaves off the fluorescent dye-labeled nucleotide from the probe during digestion of the probe hybridized to its complementary sequence in the target strand and thus separating the fluorophore from the quencher. Three sets of primers and probe were designed to detect two target regions in the RNA of SARS-CoV-2 and a region in human RNase P. The SARS-CoV-2 nCoV-NP1 probe (SEQ ID NO: 3) is labelled with dye FAM on 5′ and quencher BHQ-1 on 3′. The nCoV-NP2 (SEQ ID NO: 6) is labelled with dye Quasar 670 (detectable in CY5 channel) on 5′ and quencher BHQ-3 plus on 3′. The RNase P probe, RP (SEQ ID NO: 21) is labelled with dye CAL Fluor Orange 560 (detectable in VIC channel) on 5′ and quencher BHQ-1 on 3′ (Table 1).

The reagents and materials used in the Triplex CII-SARS-CoV-2 rRT-PCR assay can include six vials containing primers and probes for each of the viral targets (SARS-CoV-2-N1, and SARS-CoV-2-N2) (SEQ ID NOs: 1-6), three vials containing primers and probe for RNase P (SEQ ID NOs: 19-21), one vial each containing plasmid-derived in vitro transcribed RNA preparations for use as positive controls for SARS-CoV-2-N1, and SARS-CoV-2-N2 (PC-1 and PC-2, respectively), and two vials of human specimen extraction control (HSC) for use as extraction control, one vial of extracted nucleic acid from human specimen control (eHSC) to be used as a positive control in rRT-PCR for detection of human RNase P mRNA and as a negative control for detection of viral RNA, two vials of sterile distilled H₂O (NTC) to be used as non-template control, and one vial of nuclease-free water for reconstitution of primers and probes. Each Triplex CII-SARS-CoV-2 rRT-PCR assay kit provides the following reagents, which are sufficient for performing 500 reactions (Table 2).

TABLE 2
Kit components of Triplex CII-SARS-CoV-2
rRT-PCR assay (500 reactions)

| | | | | Volume |
|---|---|---|---|---|
| | Tube | Tube contents | No. | (μl) |
| 1 | nCOV-NF1 | SARS-CoV-2 forward primer for | 1 | 375 |
| | | N1 target (SEQ ID NO: 1) | | |
| 2 | nCOV-NR1 | SARS-CoV-2 reverse primer for | 1 | 375 |
| | | N1 target (SEQ ID NO: 2) | | |
| 3 | nCOV-NP1 | SARS-CoV-2 FAM Probe for N1 | 1 | 250 |
| | | target (SEQ ID NO: 3) | | |
| 4 | nCOV-NF2 | SARS-CoV-2 forward primer for | 1 | 375 |
| | | N2 target (SEQ ID NO: 4) | | |
| 5 | nCOV-NR2 | SARS-CoV-2 reverse primer for | 1 | 375 |
| | | N2 target (SEQ ID NO: 5) | | |
| 6 | nCOV-NP2 | SARS-CoV-2 Quasar 670 Probe | 1 | 250 |
| | | for N2 target (SEQ ID NO: 6) | | |
| 7 | RP-F | RNAse P forward primer | 1 | 125 |
| | | (SEQ ID NO: 19) | | |
| 8 | RP-R | RNAse P reverse primer | 1 | 125 |
| | | (SEQ ID NO: 20) | | |
| 9 | RP-P | RNAse P CAL Fluor Orange 560 | 1 | 75 |
| | | Probe (SEQ ID NO: 21) | | |
| 10 | PC1 | SARS-CoV-2 N1 target Positive | 1 | 40 |
| | | Control | | |
| 11 | PC2 | SARS-CoV-2 N2 target Positive | 1 | 40 |
| | | Control | | |
| 14 | HSC | Human specimen extraction | 2 | 250 |
| | | control | | |
| 15 | eHSC | Extracted nucleic acid from HSC | 1 | 40 |
| 16 | NTC | Nuclease free water | 1 | 750 |
| 17 | Diluent* | Nuclease free water | 1 | 750 |

Additional material for use in the assay include:

For Nucleic acid isolation

NucliSENS® easyMag® automated total nucleic acid extraction method

NucliSENS® easyMag® Magnetic Silica (bioMérieux catalog #280133)

NucliSENS® easyMag® Disposables (bioMérieux catalog #280135)

NucliSENS® easyMag® Buffer 1 (bioMérieux catalog #280130)

NucliSENS® easyMag® Buffer 2 (bioMérieux catalog #280131)

NucliSENS® easyMag® Buffer 3 (bioMérieux catalog #280132)

NucliSENS® easyMag® Lysis Buffer (bioMérieux catalog #280134)

### PCR Reagents

Molecular-grade water, nuclease-free

RNA UltraSense™ One-Step Quantitative RT-PCR System (ThermoFisher Scientific, catalog #11732927); Manual

### Equipment Required

BioMérieux NucliSENS® easyMag® (bioMérieux; catalog #280140)

Applied Biosystems™ 7500 Fast Dx Real-Time PCR Instrument (ABI cat #4406985)

Vortex mixer

Microcentrifuge

96-well cold block (and/or ice bath)

Micropipettes (2, 10, 20, 200 and 1000 μl)

Multichannel micropipette (2-20 μl)

Reaction tube racks

### Consumables

Applied Biosystems™ MicroAmp™ Fast Optical 96-Well Reaction Plate with

Barcode, 0.1 mL (Thermo Fisher cat no. #4346906)

MicroAmp™ Optical Adhesive Film (Thermo Fisher cat no. #4360954)

DNA AWAY™ (Thermo Scientific; catalog #7010)

RNase AWAY™ (Thermo Scientific; catalog #7003)

10% bleach (1:10 dilution of commercial 5.25-6.0% hypochlorite bleach)

Disposable gloves

Disposable gowns

Permanent Alcohol/Waterproof Lab Markers

Aerosol barrier sterile pipette tips for P2, P10, P20, P200, and P1000

0.5 mL and 1.5 mL reaction tubes

### Example 3—Performance of the Triplex CII-SARS-CoV-2 rRT-PCR Assay Sample Preparation

1. Aliquoted 750 ul easyMAG lysis buffer into 2 ml tubes.
2. Thoroughly vortexed swab in virus transport medium and carefully transfer 250 ul to 2 ml tube containing lysis buffer.
3. Include extraction controls containing virus transport medium only. Extraction controls should be placed at the end of each cassette.
4. Pulse vortexed three times. Quickly spun to remove droplets from the lid and leave at room temperature for 15 minutes.
5. Transferred 1 ml of swab/lysis material to a single well within the easyMAG disposable cassette.
6. Added 50 ul easyMAG magnetic silica to each well and mixed thoroughly by pipetting up and down several times.
7. Transferred cassette to the easyMAG platform
8. Installed disposable pipette tips and followed easyMAG user manual for setting up run. Elution volume was set to 50 ul.
9. At completion of run, transferred 50 ul elute to a labeled 1.7 ml tube. To expedite downstream processes, elute from each cassette can also be aliquoted to 0.2 mL 8-strip tubes.
10. Stored extracted material at 4° C. until ready to set up PCR.

### Master Mix Preparation

The master mix was prepared (Table 3) using the PCR primers (10 μM), probes (10 μM), Ultrasense RT-PCR 5× mastermix and ROX at room temperature (20° C. to 25° C.) for the appropriately sized tube as set forth in Table 3.

TABLE 3
Recipe for PCR master-mix

| | Volume to be added | Volume to be added |
|---|---|---|
| Reagent | for 1 reaction (μl) | for 100 reactions (μl) |
| Enzyme mix | 1.25 | 125 |
| 5 × mastermix | 5.00 | 500 |
| Nuclease free H₂0 | 8.40 | 840 |
| ROX | 0.50 | 50 |
| nCoV-NF1(10 μM) | 0.75 | 75 |
| nCoV-NR1 (10 μM) | 0.75 | 75 |
| nCoV-NP1 (10 μM) | 0.60 | 60 |
| nCoV-NF2 (10 μM) | 0.75 | 75 |
| nCoV-NR2 (10 μM) | 0.75 | 75 |
| nCoV-NP2 (10 μM) | 0.60 | 60 |
| RP-F (10 μM) | 0.25 | 25 |
| RP-R (10 μM) | 0.25 | 25 |
| RP-P (10 μM) | 0.15 | 15 |

20 μl of the PCR master-mix was dispensed into MicroAmp Fast Optical 96 Well Reaction Plates (0.1 mL).

### Sample Addition

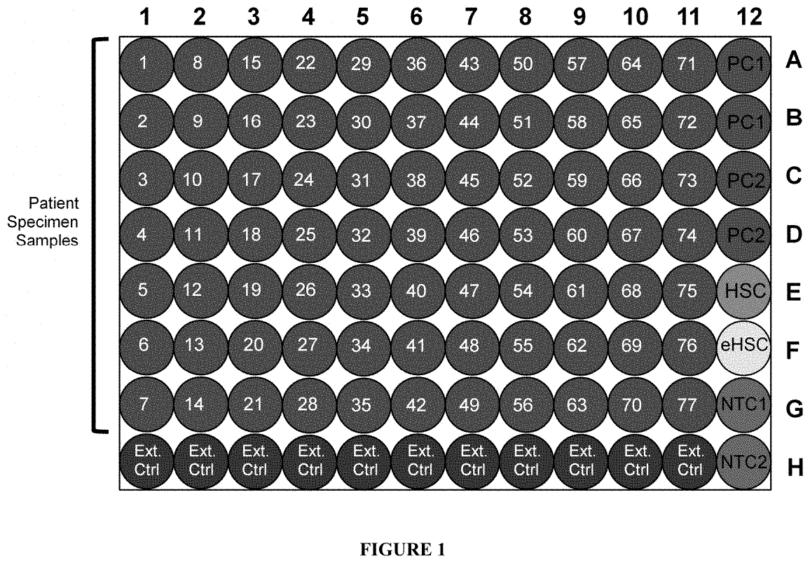

5 μl of template was added to appropriate wells. Samples included patient's specimen samples (numbered 1-77), extraction controls (labeled “Ext. Ctrl.”), positive controls for N1 and N2 targets in duplicates (PC1 and PC2), human specimen control extracted with patient's samples (labelled “HSC”), extracted human specimen control (labelled “eHSC”), and negative template controls (labelled “NTC”). See FIG. 1.

### Control Material(s) Used

A. Extraction control: Human Specimen Control (HSC)

A human cell culture preparation from Hela cells (two duplicate vials of HSC) known to contain RNase P template but negative for the SARS-CoV-2 targets. The HSC was included with each batch of test specimens to be extracted. The extracted HSC nucleic acid was included with the concurrently extracted test samples on each PCR plate and analyzed by rRT-PCR.

The HSC should generate negative results for viral targets (no fluorescent signal from the respective probes/fluorophores), but a positive result should be obtained for RNase P (fluorescence signal for VIC).

B. Internal Control: Extracted Human Specimen Control (eHSC)

Extracted total nucleic acid from a human cell culture preparation known to contain RNase P (eHSC), but negative for viral targets, was used as a control for performance of RNase P primer/probe set and PCR reagent function.

C. No Template Control: NTC

Two NTC (sterile, nuclease-free water) was run on each PCR plate.

D. SARS-CoV-2 Positive Controls: PC-1 and PC-2

In vitro transcribed synthetic RNA was used for each of SARS-CoV-2 N-gene targets. PC-1 will only be reactive for N1 target and PC-2 will only be reactive for N2 target. Providing separate positive controls for each target reduces the risk of false positives caused by carry-over contamination and mishandling of the positive control. Positive control transcripts are mixed into a background of yeast t-RNA and used as a control for performance of SARS-CoV-2 primer/probe sets and PCR reagent function. PC-1 and PC-2 were provided at approximately >50 times the limit of detection.

E. RNase P Control in Clinical Samples.

All clinical samples were tested for human RNase P, using the RP primer and probe set to control for specimen quality and as an indicator that nucleic acid resulted from the extraction process.

### Running the Assay

The sealed PCR plate containing the master mix and template were placed into a ABI 7500 platform. 4. Clicked “Plate Setup” and add three targets under “Define Targets and Samples” tab as follows:

N1 (Reporter: FAM; Quencher: None)

N2 (Reporter: CYS; Quencher: None)

RNase P (Reporter: VIC; Quencher: None)

Clicked the “Assign Targets and Samples” tab and selected all wells containing samples. Ensure that all three targets are selected for each of these wells.

Clicked run method and adjusted settings to reflect cycling conditions for this assay:
50° C. for 30 minutes;
95° C. for 10 minutes;
45 cycles of (95° C. for 15 seconds, 60° C.* for 1 minute)
   *Acquire FAM, CY5 and VIC for all sample wells.

### Results

1. At the completion of the run, the “Analysis” tab automatically opened.
2. Each target is analyzed separately by selecting either N1, N2 or RNase P from the “Target” drop-down menu.
3. Uncheck the “Threshold”, “Auto” box
4. Uncheck the “Auto Baseline” box
5. Set threshold appropriately taking into consideration the following:
6. Threshold should be set above background noise so as to exclude a non-specific increase in fluorescence.
7. A threshold of 0.005 is suggested.
8. Ensure that all curves that cross the threshold are reviewed carefully to exclude nonspecific reactivity.
9. Results can be exported using the “Export” button. Reports can be generated by clicking the “Print Report . . . ” button.
10. Positive controls should provide a Ct value of between 25-35 for their respective targets.

Signal indicative of the presence of RNA of SARS-CoV-2 in a sample extract was considered to be evidence of SARS-CoV-2 infection only if the negative control did not show viral signal and all other controls work appropriately, as described below in testing algorithm Table 4.

TABLE 4
Result Interpretations: Testing Algorithm

| SARS- | SARS- | RNase P | RNase P | RNase P | | |
|---|---|---|---|---|---|---|
| CoV-2 N1 | CoV-2 N2 | in sample | in HSC | in eHSC | | |
| positive | positive | positive | positive | positive | Interpretation* | Reporting |
| + | + | + | + | + | SARS-Cov-2 | Report |
| | | | | | RNA detected | positive |
| | | | | | by N1 and N2 | results |
| | | | | | Targets | |
| + | + | − | + | + | SARS-Cov-2 | Report |
| | | | | | RNA detected | positive |
| | | | | | by N1 and N2 | results |
| | | | | | Targets | |
| + | − | + or − | + | + | SARS-Cov-2 | Report |
| | | | | | RNA detected | positive |
| | | | | | by N1 Target | results |
| − | + | + or − | + | + | SARS-Cov-2 | Report |
| | | | | | RNA detected | positive |
| | | | | | by N2 Target | results |
| − | − | + | + | + | No SARS- | Report |
| | | | | | Cov-2 RNA | negative |
| | | | | | detected | results |
| − | − | − | + | − | Inconclusive | Repeat |
| | | | | | | assay |
| − | − | − | + | + | Invalid | Repeat |
| | | | | | | nucleic |
| | | | | | | acid |
| | | | | | | extraction |
| | | | | | | from |
| | | | | | | specimen |
| − | − | − | − | + | Invalid | Repeat |
| | | | | | | nucleic |
| | | | | | | acid |
| | | | | | | extraction |
| | | | | | | from |
| | | | | | | specimen |
| | | | | | | and HSC |

If repeat testing continues to generate negative RNAse P signal, get the new vial of HELA cells and extract the fresh HSC.

If testing produces a Ct that is greater than 39 in either N1 or N2, the sample should be re-extracted. If repeat testing continues to generate a Ct greater than 39, the sample should be reported as EQUIVOCAL and repeat sample collection is recommended.

### Quality Control—Specimen Run Controls and Acceptable Limits

Two no template controls, two positive controls (PC1 and PC2, in duplicate), HSC and eHSC controls should be included on every PCR plate along with extraction controls.
1. Negative Control: The NTC and extraction controls should not be reactive for N1 and N2. If any reactivity is detected in these wells, the run is contaminated. Nucleic acid extraction of all positive samples must be repeated. Negative samples may be reported if the N1 and N2 controls meet pass guidelines, and RNase P is detected.
2. Positive Control: Positive controls PC1 and PC2 should return a Ct value of between 25 and 35. If the Ct value is >35, the run should be interpreted with caution. If PC1 and PC2 are completely nonreactive, the run should be repeated by preparing a fresh master mix and fresh PC1 and PC2 controls.
3. Internal extraction control (RNase P): All samples should be reactive for RNase P. If samples are not reactive for N1, N2, or RNase P, this may indicate the presence of PCR inhibitors in the sample, or a failed extraction. Any nonreactive samples must be re-extracted. Samples reactive for N1 and N2, but nonreactive for RNase P can be reported as positive for SARS-CoV-2 RNA.
4. HSC: The HSC should generate negative results for viral targets (no fluorescent signal from the respective probes/fluorophores), but a positive result should be obtained for RNase P (fluorescence signal in channel VIC).
5. eHSC: Extracted total nucleic acid from a human hela cell culture preparation known to contain RNase P (fluorescence signal in channel VIC), but negative for viral targets, is used as a control for performance of primer/probe sets and PCR reagent function.
6. RNase P in sample wells: RNase P should be positive for each sample to confirm successful extraction. If RNase P is negative in some sample wells, either these individual extractions failed (e.g. machine failure for the respective sample slots), samples contain PCR inhibitors, or the samples may contain not enough RNase P for detection. If RNase P is negative for a sample but a positive viral signal is recorded and eHSC on the plate is positive for RNase P, the result for that sample can be reported. For samples without viral signal and RNase P signal, sample extraction should be repeated from a new specimen aliquot. If the samples remain negative for RNase P, the result for these samples is inconclusive. If RNase P is negative in all sample wells and for eHSC, repeat assay using fresh reagent aliquots.

### Example 4—Validation of Triplex CII-SARS-CoV-2 rRT-PCR—Limit of Detection (LoD)—Analytical Sensitivity

The assay described in Examples 2 and 3 was validated for LoD as follows.

Pre-quantified T7 RNA in vitro transcripts for SARS-COV-2 N1 and N2 targets were serially 3-fold diluted with a background of salmon sperm DNA Ong/up in 50 ul of water. 50 ul of diluted T7 RNA transcripts were spiked in 200 ul of VTM with an OP swab from a healthy control. Total 250 ul was mixed with 750 ul Easymag lysis buffer 1 and extracted according to manufacturer's instructions. 50 ul of TNA extract was eluted and 5 ul of each TNA was used in rRT-PCR reactions.

For LoD determination, each sample was extracted in triplicate and the highest dilution that had 3/3 positives was defined as tentative LoD (Table 5). LoD confirmation using full length RNA is described in the Clinical Evaluation section.

Confirmation of LoD: 20 replicates were extracted with spiked T7 RNA transcripts and used for rRT PCRs. 19/20 were positive for N1 and 20/20 were positive for N2 targets (Table 6).

TABLE 5
Determination of the tentative LoD for the Triplex CII-SARS-CoV-2 assay in oropharyngeal swab samples.

| | | Stock | Serial | | | | | | | Virus | |
|---|---|---|---|---|---|---|---|---|---|---|---|
| | | Synthetic | 3-Fold | Spiked T7 RNA | | | | | | copy | |
| | Gene | RNA Titer | Dilution | (N1; transcripts/ | PCR | PCR | PCR | Ave | Call | number | Rnase |
| Analytes | (Target) | (GEV/ul) | Factor | extraction) | Ct1 | Ct2 | Ct3 | Ct | Rate | (GEQ/ml) | P |
| SARS- | Nucleocapsid | 1 × 10*4 | 1 | 50000 | 25.7 | NP | NP | 25.7 | 1/1 | 2.00E+05 | POS |
| CoV-2 | (N1) | | 2 | 16670 | 27.6 | NP | NP | 27.6 | 1/1 | 6.67E+04 | POS |
| | | | 3 | 5560 | 29.2 | NP | NP | 29.2 | 1/1 | 2.22E+04 | POS |
| | | | 4 | 1850 | 30.8 | NP | NP | 30.8 | 1/1 | 7.40E+03 | POS |
| | | | 5 | 620 | 32.0 | 30.9 | 31.5 | 31.5 | 3/3 | 2.48E+03 | POS |
| | | | 6 | 210 | 35.3 | 32.8 | 33.2 | 33.8 | 3/3 | 8.40E+02 | POS |
| | | | 7 | 70 | 35.9 | 35.3 | 34.1 | 35.1 | 3/3 | 2.80E+02 | POS |
| | | | 8 | 25 | ND | ND | ND | ND | 0/3 | N | POS |
| | | | 9 | 10 | ND | NP | NP | ND | 0/1 | N | POS |
| | | Stock | Serial | | | | | | | Virus | |
| | | Synthetic | 3-Fold | Spiked T7 RNA | | | | | | copy | |
| | Gene | RNA Titer | Dilution | (N2; transcripts/ | PCR | PCR | PCR | Ave | Call | number | |
| Analytes | (Target) | (GEV/ul) | Factor | extraction) | Ct1 | Ct2 | Ct3 | Ct | Rate | (GEQ/ml) | Rnase P |
| SARS- | Nucleocapsid | 1 × 10*4 | 1 | 50000 | 25.4 | NP | NP | 25.4 | 1/1 | 2.00E+05 | POS |
| CoV-2 | (N2) | | 2 | 16670 | 27.7 | NP | NP | 27.7 | 1/1 | 6.67E+04 | POS |
| | | | 3 | 5560 | 29.0 | NP | NP | 29.0 | 1/1 | 2.22E+04 | POS |
| | | | 4 | 1850 | 30.9 | NP | NP | 30.9 | 1/1 | 7.40E+03 | POS |
| | | | 5 | 620 | 32.2 | 30.9 | 30.9 | 31.3 | 3/3 | 2.48E+03 | POS |
| | | | 6 | 210 | 34.1 | 33.7 | 32.6 | 33.5 | 3/3 | 8.40E+02 | POS |
| | | | 7 | 70 | 36.4 | 36.4 | 35.7 | 36.2 | 3/3 | 2.80E+02 | POS |
| | | | 8 | 25 | ND | ND | ND | ND | 0/3 | N | POS |
| | | | 9 | 10 | ND | NP | NP | ND | 0/1 | N | POS |

ND, not detected;
NP, not performed

TABLE 6
Triplex CII-SARS-CoV-2 LoD confirmation

| No. replicates | SARS-CoV-2 | SARS-CoV-2 |
|---|---|---|
| Virus concn | N1 | N2 |
| 2.8 × 10² GEQ/ml | Ct | Ct |
| Replicate 1 | 37.2 | 34.8 |
| Replicate 2 | 39.9 | 35.1 |
| Replicate 3 | 38.8 | 36.1 |
| Replicate 4 | 35.6 | 37.7 |
| Replicate 5 | 36.3 | 39.6 |
| Replicate 6 | 34.6 | 43.0 |
| Replicate 7 | 35.4 | 37.2 |
| Replicate 8 | ND | 35.6 |
| Replicate 9 | 35.9 | 35.6 |
| Replicate 10 | 36.2 | 35.4 |
| Replicate 11 | 35.9 | 36.1 |
| Replicate 12 | 35.5 | 36.1 |
| Replicate 13 | 34.9 | 36.2 |
| Replicate 14 | 34.5 | 35.6 |
| Replicate 15 | 37.0 | 41.2 |
| Replicate 16 | 35.0 | 36.3 |
| Replicate 17 | 35.0 | 35.7 |
| Replicate 18 | 36.1 | 35.5 |
| Replicate 19 | 36.5 | 36.3 |
| Replicate 20 | 35.1 | 35.9 |
| Average Ct | 36.1 | 36.7 |
| STD | 1.3 | 2.1 |
| % CV | 3.7 | 5.6 |
| Call rate | 19/20 | 20/20 |
| Call rate % | 95% | 100% |

### Example 5—Validation of Triplex CII-SARS-CoV-2 rRT-PCR—Inclusivity—Analytical Sensitivity

The assay described in Examples 2 and 3 was validated for inclusivity as follows.

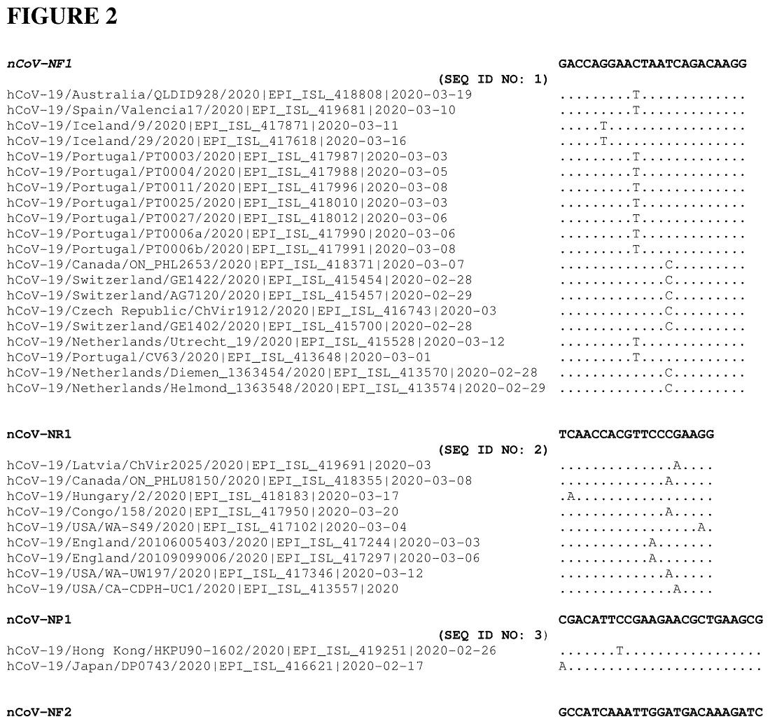

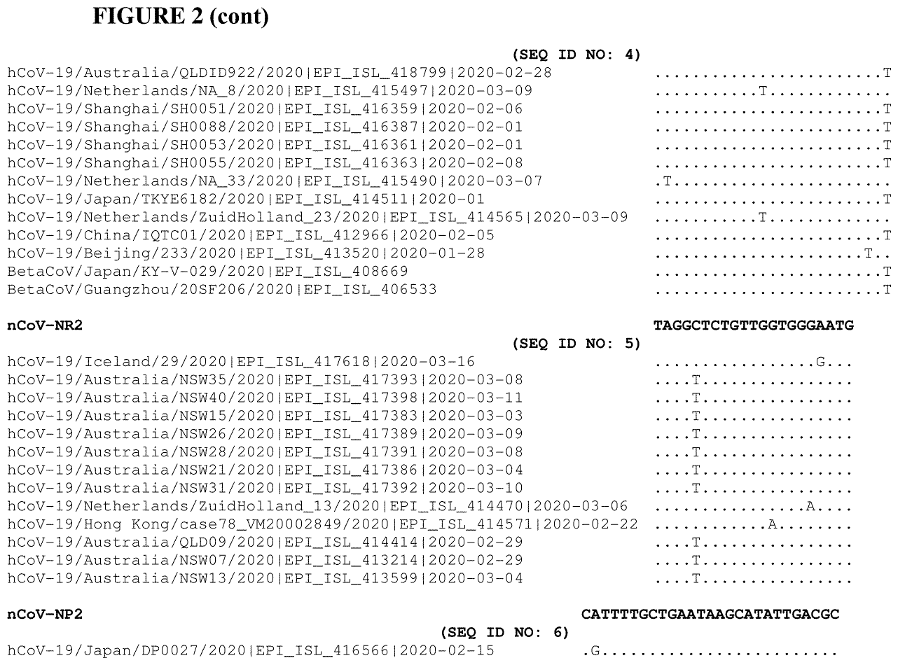

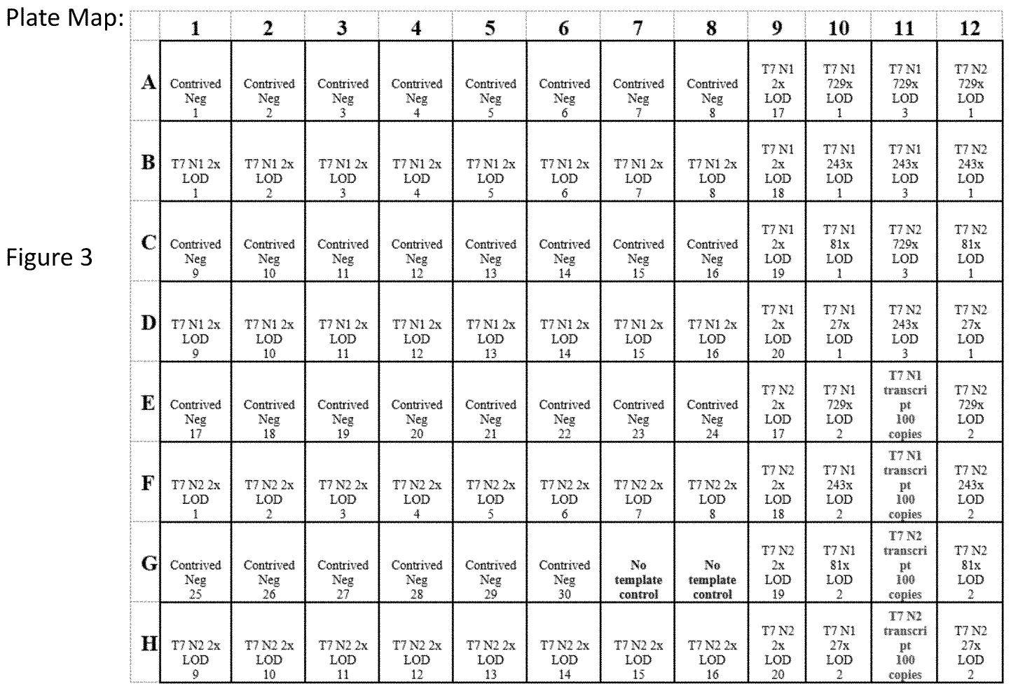

All available full length SARS-CoV-2 genomic sequences were downloaded from GISAID on Apr. 3, 2020 (n=3210) and aligned to primer and probe sequences for N1 and N2 targets. Any genomic sequences containing ambiguous bases within primer or probe binding regions were excluded. 3153/3210 (98.22%) of SARS-CoV2 genomic sequences showed 100% nucleotide identity with N1 and N2 primers and probes (Table 7). There were no genomic sequences that showed greater than one nucleotide mismatch with individual N1/N2 primers and probes (Table 7). One genomic sequence (hCoV-19/Iceland/29/2020|EPI_ISL_417618) had a single mismatch in NF1 and a single mismatch in NR2 (FIG. 2). All other genomic sequences had at most one mismatch against one of the N1/N2 primers and probes, ensuring that at least one complete primer/probe set was 100% identical.

TABLE 7
Inclusivity testing of CII-CoV-PCR assay primers and probes

| | | No mismatches | One mismatch | |
|---|---|---|---|---|
| Name | Primer/probe | (100% identity) | (94.7-96% identity) | |
| nCoV-NF1 | Forward primer | 3190/3210 (99.38%) | 20/3210 | (0.62%) |
| nCoV-NR1 | Reverse primer | 3201/3210 (99.72%) | 9/3210 | (0.28%) |
| nCoV-NP1 | Probe | 3208/3210 (99.94%) | 2/3210 | (0.06%) |
| nCoV-NF2 | Forward primer | 3197/3210 (99.60%) | 13/3210 | (0.40%) |
| nCoV-NR2 | Reverse primer | 3197/3210 (99.60%) | 13/3210 | (0.40%) |
| nCoV-NP2 | Probe | 3209/3210 (99.97%) | 1/3210 | (0.03%) |

Fifty-six SARS-CoV-2 genomic sequences were not 100% identical to primer or probe sequences (FIG. 2). There was at most a single nucleotide mismatch when comparing genomic sequences to individual primers and probes. It is anticipated that viruses with a single nucleotide mismatch and greater than 94% identity across primer and probe sequences would cross-react. In the unlikely event that the primer or probe for one target does not cross react, in 99.96% of sequences available in GISAID, the alternate target is 100% identical and would generate a positive result.

### Example 6—Validation of Triplex CII-SARS-CoV-2 rRT-PCR—Cross-Reactivity—Analytical Sensitivity

The assay described in Examples 2 and 3 was validated for cross-reactivity using in silico and in vitro assessments as follows.

Reactivity of the Triplex CII-SARS-CoV-2 rRT-PCR assay primer and probe sets were tested in silico for potential cross-reactivity with sequences of other representative respiratory viral and bacterial pathogen as indicated in the FDA_EUA-Covid19-Template document (Table 8). The specificity for the cognate sequences was assessed by in silico comparison to other agents that may cause respiratory illness using the BLAST algorithm (NCBI) (Table 9). NF2 and NR2 primers showed 96% and 100% homology with SARS CoV (2003), but NP2 did not show any homology with SARS CoV, thus, there is not expectation of any non-specific amplification with SARS-CoV. Due to absence of SARS CoV RNA, in vitro testing could not be performed. There was no match nucleotide match greater than 72% with any other organism sequence and set using the SARS-CoV-2 primer or probe sequences.

For several of the agents, in vitro rRT-PCR testing (Table 9) was also performed. Extracted nucleic acids were spiked in to 250 microliters of VTM collected with oropharyngeal swabs samples from a healthy individual and extracted using Easymag platform. No amplification was observed with any of the templates, providing no evidence of potential false positive results with the tested organisms (Table 9).

TABLE 8
Detailed in silico testing

| | | Forward | Reverse | | Forward | Reverse | |
|---|---|---|---|---|---|---|---|
| | | primer-1 | primer-1 | Probe-1 | primer-2 | primer-2 | Probe-2 |
| | | NF1 | NR1 | NP1 | NF2 | NR2 | NP2 |
| Other high priority | RefSeq Accn | XXX | XXX | XXX | XXX | XXX | XXX |
| pathogens that may | # | | | | | | |
| cause respiratory | | | | | | | |
| illness | | | | | | | |
| Human coronavirus | NC_002645.1 | XXX | XXX | XXX | XXX | XXX | XXX |
| 229E | | | | | | | |
| Human coronavirus | NC_006213.1 | XXX | XXX | XXX | XXX | XXX | XXX |
| OC43 | | | | | | | |
| Human coronavirus | NC_0065772 | XXX | XXX | XXX | XXX | XXX | XXX |
| HKU1 | | | | | | | |
| Human coronavirus | NC_005831.2 | XXX | XXX | XXX | XXX | XXX | XXX |
| NL63 | | | | | | | |
| SARS-coronavirus | NC_004718.3 | XXX | XXX | XXX | 96% | 100% | XXX |
| MERS-coronavirus | NC_019843.3 | XXX | XXX | XXX | XXX | XXX | XXX |
| Human | NC_039199.1 | XXX | XXX | XXX | XXX | XXX | XXX |
| Metapneumovirus | | | | | | | |
| (hMPV) | | | | | | | |
| Parainfluenza virus 1 | NC_003461.1 | XXX | XXX | XXX | XXX | XXX | XXX |
| Parainfluenza virus 2 | NC_003443.1 | XXX | XXX | XXX | XXX | XXX | XXX |
| Parainfluenza virus 3 | NC_001796.2 | XXX | XXX | XXX | XXX | XXX | XXX |
| Parainfluenza virus 4 | NC_021928.1 | XXX | XXX | XXX | XXX | XXX | XXX |
| Influenza A virus | NC_007366.1 | XXX | XXX | XXX | XXX | XXX | XXX |
| (A/New | NC_007367.1 | XXX | XXX | XXX | XXX | XXX | XXX |
| York/392/2004(H3N2) | NC_007368.1 | XXX | XXX | XXX | XXX | XXX | XXX |
| | NC_007369.1 | XXX | XXX | XXX | XXX | XXX | XXX |
| | NC_007370.1 | XXX | XXX | XXX | XXX | XXX | XXX |
| | NC_007371.1 | XXX | XXX | XXX | XXX | XXX | XXX |
| | NC_007372.1 | XXX | XXX | XXX | XXX | XXX | XXX |
| | NC_007373.1 | XXX | XXX | XXX | XXX | XXX | XXX |
| Influenza A virus | NC_007375.1 | XXX | XXX | XXX | XXX | XXX | XXX |
| (A/Korea/426/1968(H2N2)) | NC_007376.1 | XXX | XXX | XXX | XXX | XXX | XXX |
| | NC_007377.1 | XXX | XXX | XXX | XXX | XXX | XXX |
| | NC_007378.1 | XXX | XXX | XXX | XXX | XXX | XXX |
| | NC_007379.1 | XXX | XXX | XXX | XXX | XXX | XXX |
| | NC_007380.1 | XXX | XXX | XXX | XXX | XXX | XXX |
| | NC_007381.1 | XXX | XXX | XXX | XXX | XXX | XXX |
| | NC_007382.1 | XXX | XXX | XXX | XXX | XXX | XXX |
| | NC_007383.1 | XXX | XXX | XXX | XXX | XXX | XXX |
| Influenza A virus | NC_002016.1 | XXX | XXX | XXX | XXX | XXX | XXX |
| (A/Puerto | NC_002017.1 | XXX | XXX | XXX | XXX | XXX | XXX |
| Rico/8/1934(H1N1)) | NC_002018.1 | XXX | XXX | XXX | XXX | XXX | XXX |
| | NC_002019.1 | XXX | XXX | XXX | XXX | XXX | XXX |
| | NC_002020.1 | XXX | XXX | XXX | XXX | XXX | XXX |
| | NC_002021.1 | XXX | XXX | XXX | XXX | XXX | XXX |
| | NC_002022.1 | XXX | XXX | XXX | XXX | XXX | XXX |
| | NC_002023.1 | XXX | XXX | XXX | XXX | XXX | XXX |
| Influenza B virus | NC_002204.1 | XXX | XXX | XXX | XXX | XXX | XXX |
| (B/Lee/1940) | NC_002205.1 | XXX | XXX | XXX | XXX | XXX | XXX |
| | NC_002206.1 | XXX | XXX | XXX | XXX | XXX | XXX |
| | NC_002207.1 | XXX | XXX | XXX | XXX | XXX | XXX |
| | NC_002208.1 | XXX | XXX | XXX | XXX | XXX | XXX |
| | NC_002209.1 | XXX | XXX | XXX | XXX | XXX | XXX |
| | NC_002210.1 | XXX | XXX | XXX | XXX | XXX | XXX |
| | NC_002211.1 | XXX | XXX | XXX | XXX | XXX | XXX |
| Respiratory syncytial | NC_001803.1 | XXX | XXX | XXX | XXX | XXX | XXX |
| virus | | | | | | | |
| Chlamydia | NC_005043.1 | 69% | XXX | XXX | XXX | XXX | XXX |
| pneumoniae | | | | | | | |
| Haemophilus | NZ_LN831035 | XXX | XXX | XXX | XXX | XXX | XXX |
| influenzae | | | | | | | |
| Legionella | NZ_LR134380.1 | XXX | XXX | XXX | XXX | XXX | XXX |
| pneumophila | | | | | | | |
| Mycobacterium | NC_000962.3 | XXX | XXX | XXX | XXX | XXX | XXX |
| tuberculosis | | | | | | | |
| Streptococcus | NZ LN831051.1 | XXX | XXX | XXX | XXX | XXX | XXX |
| pneumoniae | | | | | | | |
| Streptococcus | NZ LN831034 | XXX | XXX | XXX | XXX | XXX | XXX |
| pyogenes | | | | | | | |
| Bordetella pertussis | NC_018518.1 | XXX | XXX | XXX | XXX | XXX | XXX |
| Mycoplasma | NZ CP010546.1 | XXX | XXX | XXX | XXX | XXX | XXX |
| pneumoniae | | | | | | | |
| Pneumocystis | MK984200 | XXX | XXX | XXX | XXX | XXX | XXX |
| jirovecii (PJP) | | | | | | | |
| Candida albicans | NC_018046 | XXX | XXX | XXX | XXX | XXX | XXX |
| Pseudomonas | NC_002516.2 | XXX | XXX | XXX | XXX | XXX | XXX |
| aeruginosa | | | | | | | |
| Staphylococcus | NZ_CP035288.1 | XXX | XXX | XXX | XXX | XXX | XXX |
| epidermidis | NZ_CP035289.1 | XXX | XXX | XXX | XXX | XXX | XXX |
| ASM609437v1 | NZ_CP035290.1 | XXX | XXX | XXX | XXX | XXX | XXX |
| Streptococcus | NZ_LR134274.1 | XXX | XXX | XXX | XXX | XXX | XXX |
| salivarius | | | | | | | |
| Human enterovirus D | NC_001430.1 | XXX | XXX | XXX | XXX | XXX | XXX |
| Human enterovirus B | NC_001472.1 | XXX | XXX | XXX | XXX | XXX | XXX |
| Human rhinovirus 14 | NC_001490.1 | XXX | XXX | XXX | XXX | XXX | XXX |
| Human enterovirus A | NC_001612.1 | XXX | XXX | XXX | XXX | XXX | XXX |
| Human rhinovirus 89 | NC_001617.1 | XXX | XXX | XXX | XXX | XXX | XXX |
| Bovine enterovirus | NC_001859.1 | XXX | XXX | XXX | XXX | XXX | XXX |
| Poliovirus | NC_002058.3 | XXX | XXX | XXX | XXX | XXX | XXX |
| Porcine teschovirus 1 | NC_003985.1 | XXX | XXX | XXX | XXX | XXX | XXX |
| Porcine sapelovirus 1 | NC_003987.1 | XXX | XXX | XXX | XXX | XXX | XXX |
| Simian enterovirus A | NC_003988.1 | XXX | XXX | XXX | XXX | XXX | XXX |
| Porcine enterovirus 9 | NC_004441.1 | XXX | XXX | XXX | XXX | XXX | XXX |
| strain UKG/410/73 | | | | | | | |
| polyprotein gene, | | | | | | | |
| Avian sapelovirus | NC_006553.1 | XXX | XXX | XXX | XXX | XXX | XXX |
| Possum enterovirus | NC_008714.1 | XXX | XXX | XXX | XXX | XXX | XXX |
| W1 | | | | | | | |
| Human rhinovirus C | NC_009996.1 | XXX | XXX | XXX | XXX | XXX | XXX |
| Enterovirus J strain | NC_010415.1 | XXX | XXX | XXX | XXX | XXX | XXX |
| 1631 | | | | | | | |
| Enterovirus J strain | NC_013695.1 | XXX | XXX | XXX | XXX | XXX | XXX |
| N203 | | | | | | | |
| Enterovirus F strain | NC_021220.1 | XXX | XXX | XXX | XXX | XXX | XXX |
| BEV-261 polyprotein | | | | | | | |
| gene, complete | | | | | | | |
| Enterovirus sp. isolate | NC_024073.1 | XXX | XXX | XXX | XXX | XXX | XXX |
| CPML_8109/08 | | | | | | | |
| Yak enterovirus | NC_029854.1 | XXX | XXX | XXX | XXX | XXX | XXX |
| strain SWUN-AB001 | | | | | | | |
| Enterovirus SEV-gx | NC_029905.1 | XXX | XXX | XXX | XXX | XXX | XXX |
| Human enterovirus | NC_030454.1 | XXX | XXX | XXX | XXX | XXX | XXX |
| strain V13-0285, | | | | | | | |
| partial genome | | | | | | | |
| Ovine adenovirus A | AC_000001.1 | XXX | XXX | XXX | XXX | XXX | XXX |
| Bovine adenovirus B | AC_000002.1 | XXX | XXX | XXX | XXX | XXX | XXX |
| Canine adenovirus 1 | AC_000003.1 | XXX | XXX | XXX | XXX | XXX | XXX |
| Duck adenovirus A | AC_000004.1 | XXX | XXX | XXX | XXX | XXX | XXX |
| Human | AC_000005.1 | XXX | XXX | XXX | XXX | XXX | XXX |
| mastadenovirus A | | | | | | | |
| Human adenovirus D | AC_000006.1 | XXX | XXX | XXX | XXX | XXX | XXX |
| Human adenovirus 2 | AC_000007.1 | XXX | XXX | XXX | XXX | XXX | XXX |
| Human adenovirus 5 | AC_000008.1 | XXX | XXX | XXX | XXX | XXX | XXX |
| Porcine adenovirus C | AC_000009.1 | XXX | XXX | XXX | XXX | XXX | XXX |
| Simian adenovirus 21 | AC_000010.1 | XXX | XXX | XXX | XXX | XXX | XXX |
| Simian adenovirus 25 | AC_000011.1 | XXX | XXX | XXX | XXX | XXX | XXX |
| Murine adenovirus A | AC_000012.1 | XXX | XXX | XXX | XXX | XXX | XXX |
| Fowl adenovirus D | AC_000013.1 | XXX | XXX | XXX | XXX | XXX | XXX |
| Fowl adenovirus A | AC_000014.1 | XXX | XXX | XXX | XXX | XXX | XXX |
| Turkey adenovirus A | AC_000016.1 | XXX | XXX | XXX | XXX | XXX | XXX |
| Human adenovirus | AC_000017.1 | XXX | XXX | XXX | XXX | XXX | XXX |
| type 1 | | | | | | | |
| Human adenovirus | AC_000018.1 | XXX | XXX | XXX | XXX | XXX | XXX |
| type 7 | | | | | | | |
| Human adenovirus | AC_000019.1 | XXX | XXX | XXX | XXX | XXX | XXX |
| type 35 | | | | | | | |
| Canine adenovirus | AC_000020.1 | XXX | XXX | XXX | XXX | XXX | XXX |
| type 2 | | | | | | | |
| Porcine adenovirus 3 | AC_000189.1 | XXX | XXX | XXX | XXX | XXX | XXX |
| Tree shrew | AC_000190.1 | XXX | XXX | XXX | XXX | XXX | XXX |
| adenovirus 1 | | | | | | | |
| Bovine adenovirus A | AC_000191.1 | XXX | XXX | XXX | XXX | XXX | XXX |
| Fowl adenovirus D | NC_000899.1 | XXX | XXX | XXX | XXX | XXX | XXX |
| Murine adenovirus A | NC_000942.1 | XXX | XXX | XXX | XXX | XXX | XXX |
| Human adenovirus C | NC_001405.1 | XXX | XXX | XXX | XXX | XXX | XXX |
| Human adenovirus F | NC_001454.1 | XXX | XXX | XXX | XXX | XXX | XXX |
| Human adenovirus A | NC_001460.1 | XXX | XXX | XXX | XXX | XXX | XXX |
| Fowl adenovirus A | NC_001720.1 | XXX | XXX | XXX | XXX | XXX | XXX |
| Canine adenovirus | NC_001734.1 | XXX | XXX | XXX | XXX | XXX | XXX |
| Duck adenovirus A | NC_001813.1 | XXX | XXX | XXX | XXX | XXX | XXX |
| Bovine adenovirus 3 | NC_001876.1 | XXX | XXX | XXX | XXX | XXX | XXX |
| complete genome | | | | | | | |
| Hemorrhagic enteritis | NC_001958.1 | XXX | XXX | XXX | XXX | XXX | XXX |
| virus | | | | | | | |
| Frog adenovirus 1 | NC_002501.1 | XXX | XXX | XXX | XXX | XXX | XXX |
| Bovine adenovirus | NC_002513.1 | XXX | XXX | XXX | XXX | XXX | XXX |
| type 2 | | | | | | | |
| Bovine adenovirus D | NC_002685.2 | XXX | XXX | XXX | XXX | XXX | XXX |
| Porcine adenovirus 5 | NC_002702.1 | XXX | XXX | XXX | XXX | XXX | XX |
| Human adenovirus E | NC_003266.2 | XXX | XXX | XXX | XXX | XXX | XX |
| Garlic virus A | NC_003375.1 | XXX | XXX | XXX | XXX | XXX | XX |
| Ovine adenovirus 7 | NC_004037.2 | XXX | XXX | XXX | XXX | XXX | XXX |
| Bovine adeno- | NC_005889.1 | XXX | XXX | XXX | XXX | XXX | XXX |
| associated virus | | | | | | | |
| Simian adenovirus 3 | NC_006144.1 | XXX | XXX | XXX | XXX | XXX | XXX |
| Macacine herpesvirus | NC_006150.1 | XXX | XXX | XXX | XXX | XXX | XXX |
| 3 | | | | | | | |
| Simian adenovirus 1 | NC_006879.1 | XXX | XXX | XXX | XXX | XXX | XXX |
| Snake adenovirus | NC_009989.1 | XXX | XXX | XXX | XXX | XXX | XXX |
| Human adenovirus D | NC_010956.1 | XXX | XXX | XXX | XXX | XXX | XXX |
| Rhizobium phage 16- | NC_011103.1 | XXX | XXX | XXX | XXX | XXX | XXX |
| 3 | | | | | | | |
| Human adenovirus | NC_011202.1 | XXX | XXX | XXX | XXX | XXX | XX |
| B2 | | | | | | | |
| Human adenovirus | NC_011203.1 | XXX | XXX | XXX | XXX | XXX | XX |
| B1 | | | | | | | |
| Murine adenovirus 3 | NC_012584.1 | XXX | XXX | XXX | XXX | XXX | XXX |
| Cercopithecine | NC_012783.2 | XXX | XXX | XXX | XXX | XXX | XXX |
| herpesvirus 5 strain | | | | | | | |
| 2715 | | | | | | | |
| Human adenovirus 54 | NC_012959.1 | XXX | XXX | XXX | XXX | XXX | XXX |
| Turkey adenovirus 1 | NC_014564.2 | XXX | XXX | XXX | XXX | XXX | XXX |
| Murine adenovirus 2 | NC_014899.1 | XXX | XXX | XXX | XXX | XXX | XXX |
| Fowl adenovirus E | NC_014969.1 | XXX | XXX | XXX | XXX | XXX | XXX |
| Japanese eel | NC_015123.1 | XXX | XXX | XXX | XXX | XXX | XXX |
| endothelial cells- | | | | | | | |
| infecting virus | | | | | | | |
| Simian adenovirus 49 | NC_015225.1 | XXX | XXX | XXX | XXX | XXX | XXX |
| Fowl adenovirus C | NC_015323.1 | XXX | XXX | XXX | XXX | XXX | XXX |
| Raptor adenovirus A | NC_015455.1 | XXX | XXX | XXX | XXX | XXX | XXX |
| Bat adenovirus 2 | NC_015932.1 | XXX | XXX | XXX | XXX | XXX | XX |
| South polar skua | NC_016437.1 | XXX | XXX | XXX | XXX | XXX | XXX |
| adenovirus-1 | | | | | | | |
| Bat adenovirus TJM | NC_016895.1 | XXX | XXX | XXX | XXX | XXX | XXX |
| Chimpanzee | NC_017825.1 | XXX | XXX | XXX | XXX | XXX | XXX |
| adenovirus Y25 | | | | | | | |
| Goose adenovirus 4 | NC_017979.1 | XXX | XXX | XXX | XXX | XXX | XXX |
| Bovine adenovirus 6 | NC_020074.1 | XXX | XXX | XXX | XXX | XXX | XXX |
| strain 671130 | | | | | | | |
| Simian adenovirus 20 | NC_020485.1 | XXX | XXX | XXX | XXX | XXX | XXX |
| strain ATCC VR-541 | | | | | | | |
| Titi monkey | NC_020487.1 | XXX | XXX | XXX | XXX | XXX | XXX |
| adenovirus ECC-2011 | | | | | | | |
| Simian adenovirus C | NC_021168.1 | XXX | XXX | XXX | XXX | XXX | XXX |
| isolate BaAdV-2 | | | | | | | |
| Fowl adenovirus 5 | NC_021221.1 | XXX | XXX | XXX | XXX | XXX | XXX |
| strain 340 | | | | | | | |
| Pandoravirus dulcis | NC_021858.1 | XXX | 72% | XXX | XXX | XXX | XXX |
| Simian adenovirus 18 | NC_022266.1 | XXX | XXX | XXX | XXX | XXX | XXX |
| Turkey adenovirus 4 | NC_022612.1 | XXX | XXX | XXX | XXX | XXX | XXX |
| isolate TNI1 | | | | | | | |
| Turkey adenovirus 5 | NC_022613.1 | XXX | XXX | XXX | XXX | XXX | XXX |
| isolate 1277BT | | | | | | | |
| Ralstonia phage | NC_022915.1 | XXX | XXX | XXX | XXX | XXX | XXX |
| RSK1 DNA | | | | | | | |
| California sea lion | NC_024150.1 | XXX | XXX | XXX | XXX | XXX | XXX |
| adenovirus 1 strain | | | | | | | |
| Zc11-030 | | | | | | | |
| Pigeon adenovirus 1 | NC_024474.1 | XXX | XXX | XXX | XXX | XXX | XXX |
| complete genome, | | | | | | | |
| strain IDA4 | | | | | | | |
| Duck adenovirus 2 | NC_024486.1 | XXX | XXX | XXX | XXX | XXX | XXX |
| strain GR | | | | | | | |
| Lizard adenovirus 2 | NC_024684.1 | XXX | XXX | XXX | XXX | XXX | XXX |
| isolate 23-06 | | | | | | | |
| Simian adenovirus | NC_025678.1 | XXX | XXX | XXX | XXX | XXX | XXX |
| DM-2014 isolate | | | | | | | |
| 23336 | | | | | | | |
| Psittacine adenovirus | NC_025962.1 | XXX | XXX | XXX | XXX | XXX | XXX |
| 3 isolate | | | | | | | |
| HKU/Parrot19 | | | | | | | |
| Equine adenovirus 2 | NC_027705.1 | XXX | XXX | XXX | XXX | XXX | XXX |
| isolate | | | | | | | |
| EAdV2.385/75.9 | | | | | | | |
| Skunk adenovirus | NC_027708.1 | XXX | XXX | XXX | XXX | XXX | XXX |
| PB1 | | | | | | | |
| Simian adenovirus 13 | NC_028103.1 | XXX | XXX | XXX | XXX | XXX | XXX |
| strain P-9 | | | | | | | |
| Simian adenovirus 16 | NC_028105.1 | XXX | XXX | XXX | XXX | XXX | XXX |
| strain C-8 | | | | | | | |
| Simian adenovirus 19 | NC_028107.1 | XXX | XXX | XXX | XXX | XXX | XXX |
| strain AA153 | | | | | | | |
| Simian adenovirus 8 | NC_028113.1 | XXX | XXX | XXX | XXX | XXX | XXX |
| strain P-5 | | | | | | | |
| Unidentified | NC_030116.1 | XXX | XXX | XXX | XXX | XXX | XXX |
| adenovirus isolate | | | | | | | |
| CSPAdV 2 | | | | | | | |
| Tokyovirus A1 DNA, | NC_030230.1 | XXX | XXX | XXX | XXX | XXX | XXX |
| nearly complete | | | | | | | |
| genome | | | | | | | |
| Equine adenovirus 1 | NC_030792.1 | XXX | XXX | XXX | XXX | XXX | XXX |
| strain M1 | | | | | | | |
| Pigeon adenovirus 2 | NC_031503.1 | XXX | XXX | XXX | XXX | XXX | XXX |
| isolate YPDS-Y- | | | | | | | |
| V1.A19.11-2013 | | | | | | | |

XXX, no identity shared with reference sequences using BLASTn alignments

TABLE 9
In vitro testing of organisms with N gene primers and probes

| | | | | Triplex |
|---|---|---|---|---|
| | | | | CII-SARS-CoV-2 |
| | | | | rRT-PCR |
| No | Agent | Source | Titer | assay |
| 1 | Haemophilus influenzae | ATCC 10211 | NA* | ND* |
| 2 | Mycobacterium tuberculosis | Clincal specimen | 2.10E+04 | ND* |
| 3 | Streptococcus pneumoniae | NR-20805 | 2.80E+05 | ND* |
| 4 | Streptococcus pyogenes | NR-50136 | NA* | ND* |
| 5 | Bordetella pertussis | NR-42457 | 4.32E+05 | ND* |
| 6 | Candida albicans | ATCC ® | 4.97E+04 | ND* |
| | | MYA2876 | | |
| 7 | Pseudomonas aeruginosa | NR-50573 | NA* | ND* |
| 8 | Human CoV 229E | ATCC VR-740 | ~1.00E+08 | ND* |
| 9 | Human CoV OC43 | ATCC ® | ~1.00E+05 | ND* |
| | | VR-1558 | | |
| 10 | Human CoV HKU1 | ATCC | ~1.00E+05 | ND* |
| | | VR-3262SD | | |
| 11 | Human CoV NL63 | ATCC | ~1.00E+05 | ND* |
| | | VR-3263SD | | |
| 12 | Human adenovirus 3 | ATCC ® | 4.60E+04 | ND* |
| | | VR-847D ™ | | |
| 13 | Human Metapneumovirus | clinical isolate | 3.30E+03 | ND* |
| | (hMPV) | | | |
| 14 | Parainfluenza virus - 1 | ATCC ® VR-94 | 4.88E+03 | ND* |
| 15 | Parainfluenza virus - 2 | ATCC ® VR-92D | 2.89E+04 | ND* |
| 16 | Parainfluenza virus - 3 | ATCC ® | 1.29E+05 | ND* |
| | | VR-1782 | | |
| 17 | Influenza A Strain | Stored virus | 1.20E+04 | ND* |
| | A/Panama/2007/99 | culture | | |
| 18 | Influenza B/Malaysia/2506/2004 | Stored virus | 7.50E+05 | ND* |
| | | culture | | |
| 19 | Enterovirus (EVD68) | VR-1076 | 4.45E+05 | ND* |
| | | (ATCC) | | |
| 20 | Respiratory syncytial virus | VR-1540 | 1.95E+05 | ND* |
| | (RSV) | (ATCC) | | |
| 21 | Rhinovirus | HRV16 (ATCC) | 5.00E+05 | ND* |
| 22 | Enterovirus (EVA71) | Tainan -4643 | 4.90E+05 | ND* |
| | | (BEI) | | |
| 23 | Pooled human nasal wash | NA | NA | ND* |

NA* Bacterial load for these agents is not available but all were obtained from ATCC and used in other HTS project, and provided good coverage and depth.
ND* we did not detect any amplification for SARS-CoV-2 RNA using Triplex CII-SARS-CoV-2 rRT-PCR assay

### Example 7—Clinical Evaluation of Triplex CII-SARS-CoV-2 rRT-PCR

Contrived positive individual oropharyngeal swab samples in VTM were prepared by spiking with N1 or N2 RNA transcripts. Thirty non-spiked individual samples were also extracted. Nucleic acid extraction was performed on the EasyMag platform. PCR for reactive and non-reactive samples were performed on a single plate, along with appropriate no template and positive controls. The PCR platemap shown in Figure A was utilized for tracking purposes only. The analyst was blinded to the content of each sample.

The counts for the contrived positive samples are in Table 10 and the counts for the contrived negative samples are in Table 11.

TABLE 10
Contrived positive OP samples for SARS-CoV-2 assay evaluation
using in vitro transcribed RNA transcripts

| | N1-Ct measured in contrived OP | | | | | N2-Ct measured in contrived OP | | | | |
|---|---|---|---|---|---|---|---|---|---|---|
| | 2X | 27X | 81X | 243X | 729X | 2X | 27X | 81X | 243X | 729X |
| | LoD | LoD | LoD | LoD | LoD | LoD | LoD | LoD | LoD | LoD |
| 1 | 33.1 | 29.8 | 28.5 | 27.1 | 25.5 | 32.8 | 30.4 | 28.2 | 27.0 | 24.6 |
| 2 | 32.4 | 30.5 | 28.9 | 27.1 | 25.2 | ND* | 30.0 | 28.4 | 27.6 | 24.7 |
| 3 | 33.6 | | | 26.5 | 25.3 | 35.4 | | | 27.3 | 25.3 |
| 4 | 33.3 | | | | | 32.5 | | | | |
| 5 | 32.3 | | | | | 33.4 | | | | |
| 6 | 34.1 | | | | | 32.8 | | | | |
| 7 | 32.6 | | | | | 32.8 | | | | |
| 8 | 32.7 | | | | | 33.2 | | | | |
| 9 | 32.6 | | | | | 32.6 | | | | |
| 10 | 31.7 | | | | | 32.7 | | | | |
| 11 | 32.5 | | | | | 34.6 | | | | |
| 12 | 34.2 | | | | | 32.2 | | | | |
| 13 | 31.8 | | | | | 32.8 | | | | |
| 14 | 33.4 | | | | | 32.7 | | | | |
| 15 | 32.5 | | | | | 32.7 | | | | |
| 16 | 33.4 | | | | | 32.7 | | | | |
| 17 | ND | | | | | ND | | | | |
| 18 | 32.7 | | | | | 33.2 | | | | |
| 19 | 32.5 | | | | | 32.8 | | | | |
| 20 | 32.7 | | | | | 32.3 | | | | |
| Average | 32.8 | 30.2 | 28.7 | 26.9 | 25.3 | 33.0 | 30.2 | 28.3 | 27.3 | 24.9 |
| Ct | | | | | | | | | | |
| Call rate | 19/20 | 2/2 | 2/2 | 3/3 | 3/3 | 18/20 | 2/2 | 2/2 | 3/3 | 3/3 |
| Call % | 95% | 100% | 100% | 100% | 100% | 90% | 100% | 100% | 100% | 100% |

*Genuine low reactive curve; positive using deltaRn threshold of 0.002.

TABLE 11
Contrived negative OP samples for SARS-CoV-2 assay evaluation

| | PCR measured in OP swab | | |
|---|---|---|---|
| | N1 | N2 | RNaseP |
| Sample | Ct | Ct | Ct |
| 1 | ND | ND | 27.6 |
| 2 | ND | ND | 24.1 |
| 3 | ND | ND | 23.3 |
| 4 | ND | ND | 28.6 |
| 5 | ND | ND | 26.0 |
| 6 | ND | ND | 28.2 |
| 7 | ND | ND | 27.0 |
| 8 | ND | ND | 27.1 |
| 9 | ND | ND | 31.8 |
| 10 | ND | ND | 24.8 |
| 11 | ND | ND | 27.1 |
| 12 | ND | ND | 23.4 |
| 13 | ND | ND | 25.9 |
| 14 | ND | ND | 27.5 |
| 15 | ND | ND | 25.5 |
| 16 | ND | ND | 28.1 |
| 17 | ND | ND | 26.0 |
| 18 | ND | ND | 28.1 |
| 19 | ND | ND | 31.3 |
| 20 | ND | ND | 25.0 |
| 21 | ND | ND | 23.8 |
| 22 | ND | ND | 28.3 |
| 23 | ND | ND | 25.6 |
| 24 | ND | ND | 27.6 |
| 25 | ND | ND | 28.4 |
| 26 | ND | ND | 24.5 |
| 27 | ND | ND | 26.0 |
| 28 | ND | ND | 27.5 |
| 29 | ND | ND | 25.9 |
| 30 | ND | ND | 23.7 |
| Average | NA | NA | |
| Ct | | | |
| Call rate | 0/30 | 0/30 | |
| Call % | 100% | 100% | |

The analytical sensitivity (LoD) using full length viral genomic RNA was evaluated as follows. Twenty individual oropharyngeal swab samples were spiked with full length viral RNA sourced from infected Vero E6 cells (2019-nCoV/USA-WA1/2020; accession MN985325 grown in Vero E6 Cat #ATCC® CRL-1586™) and used for rRT-PCRs. Samples were extracted on the EasyMag platform. Individual swab samples were spiked with less than 2× LoD. PCR was performed in duplicate and average Cts are shown in Table 12. 20/20 were positive for N1 and 20/20 were positive for N2 targets; 1/20 were discrepant for N2 (Table 12).

TABLE 12
Contrived positive OP samples for SARS-CoV-2
assay evaluation using full length viral RNA
(2019-nCoV/USA-WA1/2020; accession MN985325)

| | PCR measured in OP swab | | |
|---|---|---|---|
| | N1 | N2 | Rnase P |
| Sample | Ct | Ct | Ct |
| 1 | 32.6 | 33.3 | 32.2 |
| 2 | 33.1 | 36.0 | 27.0 |
| 3 | 32.6 | 33.6 | 32.1 |
| 4 | 33.4 | 35.5 | 28.3 |
| 5 | 33.2 | 34.1 | 30.1 |
| 6 | 33.0 | 35.2 | 29.1 |
| 7 | 32.6 | 34.4 | 32.0 |
| 8 | 32.7 | 33.8 | 33.4 |
| 9 | 32.7 | 33.9 | 32.6 |
| 10 | 32.8 | 34.0 | 29.8 |
| 11 | 32.9 | 34.2 | 32.6 |
| 12 | 32.9 | 34.4 | 28.7 |
| 13 | 32.8 | 34.3 | 31.0 |
| 14 | 32.2 | 33.3 | 34.2 |
| 15 | 33.3 | 35.9 | 29.2 |
| 16 | 32.7 | 33.7 | 32.1 |
| 17 | 32.9 | 33.9 | 30.6 |
| 18 | 32.6 | 33.9 | 34.3 |
| 19 | 33.1 | 33.9* | 30.3 |
| 20 | 32.9 | 36.6 | 31.6 |
| Average | 32.8 | 34.4 | N/A |
| Ct | | | |
| Call rate | 20/20 | 20/20* | N/A |
| Call % | 100% | 100% | N/A |

*Sample 19 was discrepant for SARS-CoV-2 N2 target in duplicate PCRs

### Example 8—Quadraplex CII-SARS-CoV-2 rRT-PCR

The Quadraplex CII-SARS-CoV-2 rRT-PCR assay is intended for the qualitative detection of nucleic acid from the SARS-CoV-2 in nasopharyngeal (NPS) and oropharyngeal (OPS) swabs collected from individuals with suspected COVID-19. Results are for the detection and identification of SARS-CoV-2 RNA and influenza RNA. The SARS-CoV-2 RNA is generally detectable in respiratory specimens during the acute phase of infection. Positive results are indicative of active infection with SARS-CoV-2 or influenza. Other sample which can be used in the assay include but are not limited to nasal swabs, feces and saliva.

The Quadraplex CII-SARS-CoV-2 rRT-PCR assay uses one primer-probe sets to detect the nucleocapsid (N) gene of SARS-CoV-2 (SEQ ID NOs: 1-3), one primer-probe set to detect the matrix gene of influenza A (SEQ ID NOs: 13-15), one primer-probe set to detect the matrix gene of influenza B (SEQ ID NOs: 16-18) and a primer-probe set targeting the human RNase P housekeeping gene (SEQ ID NOs: 19-21) (Table 1). The Quadraplex CII-SARS-CoV-2 rRT-PCR assay can detect four targets (SARS-CoV-2 N gene, influenza A matrix gene, influenza B matrix gene, and human RNAse P) simultaneously.

The Quadraplex CII-SARS-CoV-2 rRT-PCR assay is a one-step rRT-PCR test. The assay includes primers and dual-labeled probes to be used in the in vitro qualitative detection of isolated RNA from SARS-CoV-2 and influenza and the host control transcript RNase P from clinical specimens. The assay is a real-time reverse transcription polymerase chain reaction (rRT-PCR) test. The SARS-CoV-2 primer and probe set were designed to detect RNA from SARS-CoV-2 in respiratory specimens from patients as recommended for testing by public health authority guidelines. Dual-labeled probes are linear oligonucleotides, incorporating a fluorophore and quencher covalently attached to the 5′ and 3′ nucleotides of the oligonucleotide, respectively. Fluorescence signal is generated through the 5′ exonuclease activity of Taq polymerase, which cleaves off the fluorescent dye-labeled nucleotide from the probe during digestion of the probe hybridized to its complementary sequence in the target strand and thus separating the fluorophore from the quencher.

Four sets of primers and probe were designed to detect RNA of SARS-CoV-2, influenza A, influenza B, and a region in human RNase P. The SARS-CoV-2 nCoV-NP1 probe (SEQ ID NO: 3) is labelled with dye FAM on 5′ and quencher BHQ-1 on 3′. The influenza A probe (SEQ ID NO: 15) is labelled with dye CAL Flour Orange 560 on 5′ and quencher BHQ-1 plus on 3′. The influenza B probe (SEQ ID NO: 18) is labelled with dye CAL Flour Red 610 on 5′ and quencher BHQ-2 plus on 3′. The RNase P probe, RP (SEQ ID NO: 21) is labelled with dye Quasar 670 on 5′ and quencher BHQ-3 on 3′ (Table 1).

The reagents and materials used in the Quadraplex CII-SARS-CoV-2 rRT-PCR assay can include nine vials containing primers and probes for each of the viral targets (SARS-CoV-2-N1) (SEQ ID NOs: 1-3), Influenza A (SEQ ID NOs: 13-15), influenza B (SEQ ID NOs: 16-18) and three vials containing primers and probe for RNase P (SEQ ID NOs: 19-21), one vial each containing plasmid-derived in vitro transcribed RNA preparations for use as positive controls for SARS-CoV-2-N1, influenza A and influenza B, and two vials of human specimen extraction control (HSC) for use as extraction control, one vial of extracted nucleic acid from human specimen control (eHSC) to be used as a positive control in rRT-PCR for detection of human RNase P mRNA and as a negative control for detection of viral RNA, two vials of sterile distilled H₂O (NTC) to be used as non-template control, and one vial of nuclease-free water for reconstitution of primers and probes. Each Quadraplex CII-SARS-CoV-2 rRT-PCR assay kit provides the following reagents, which are sufficient for performing 500 reactions (Table 13).

TABLE 13
Kit components of Quadraplex CII-SARS-CoV-2
rRT-PCR rRT-PCR assay (500 reactions)

| | | | | Volume |
|---|---|---|---|---|
| | Tube | Tube contents | No. | (μl) |
| 1 | nCOV-NF1 | SARS-CoV-2 forward primer for | 1 | 375 |
| | | N1 target (SEQ ID NO: 1) | | |
| 2 | nCOV-NR1 | SARS-CoV-2 reverse primer for | 1 | 375 |
| | | N1 target (SEQ ID NO: 2) | | |
| 3 | nCOV-NP1 | SARS-CoV-2 FAM Probe for N1 | 1 | 250 |
| | | target (SEQ ID NO: 3) | | |
| 4 | Influenza- | Influenza A forward primer | 1 | 375 |
| | A-MF | (SEQ ID NO: 13) | | |
| 5 | Influenza- | Influenza A reverse primer | 1 | 375 |
| | A-MR | (SEQ ID NO: 14) | | |
| 6 | Influenza- | Influenza A CAL Fluor Orange | 1 | 250 |
| | A-MP | 560 Probe (SEQ ID NO: 15) | | |
| 7 | Influenza- | Influenza B forward primer | 1 | 375 |
| | A-MF | (SEQ ID NO: 16) | | |
| 8 | Influenza- | Influenza B reverse primer | 1 | 375 |
| | A-MR | (SEQ ID NO: 17) | | |
| 9 | Influenza- | Influenza B CAL Fluor Red | 1 | 250 |
| | A-MP | 610 Probe (SEQ ID NO: 18) | | |
| 10 | RP-F | RNAse P forward primer | 1 | 125 |
| | | (SEQ ID NO: 19) | | |
| 11 | RP-R | RNAse P reverse primer | 1 | 125 |
| | | (SEQ ID NO: 20) | | |
| 12 | RP-P | RNAse P CAL Fluor Orange | 1 | 75 |
| | | 560 Probe (SEQ ID NO: 21) | | |
| 13 | PC1 | SARS-CoV-2 N1 target | 1 | 40 |
| | | Positive Control | | |
| 14 | PC2 | Influenza A Positive Control | 1 | 40 |
| 15 | PC3 | Influenza B Positive Control | 1 | 40 |
| 16 | HSC | Human specimen extraction | 2 | 250 |
| | | control | | |
| 17 | eHSC | Extracted nucleic acid | 1 | 40 |
| | | from HSC | | |
| 18 | NTC | Nuclease free water | 1 | 750 |
| 19 | Diluent* | Nuclease free water | 1 | 750 |

The Quadraplex CII-SARS-CoV-2 rRT-PCR assay uses the same additional materials and methods as those used for the Triplex CII-SARS-CoV-2 rRT-PCR (see Examples 2 and 3).

Validations, similar to the ones for the Triplex CII-SARS-CoV-2 rRT-PCR, showed the sensitivity and specificity of the primers and probes for the SARS-CoV-2 virus and influenza A and B virus (Examples 9 and 10).

Using clinical samples, showed the assay can differentially detect SARS-CoV-2 virus and influenza A and B virus (Example 11).

### Example 9—Validation of Quadraplex CII-SARS-CoV-2 rRT-PCR—Limit of Detection (LoD)—Analytical Sensitivity

The assay described in Example 8 was validated for LoD as follows.

Quantified virus stocks were serially diluted, extracted and tested in triplicate with the Quadruplex CII-SARS-Cov2 assay. The lowest concentrations detected in all three replicates for each agent are highlighted in bold in Table 14.

### Limit of Detection of Quadruplex CII-SARS-CoV-2 Assay

| | | | Detection limit | | | | | |
|---|---|---|---|---|---|---|---|---|
| | Gene | | viral copies/PCR | Detection limit | Detection | PCR | PCR | PCR |
| Virus | target | Source | reaction | virus/extraction | limit virus/ml | Ct 1 | Ct2 | Ct 3 |
| Influenza | Matrix | Quantified virus | 2.0E+02 | 4000 | 2.00E+04 | 32.99 | 33.48 | 33.71 |
| A | | stock | | | | | | |
| | | Quantified virus | 68.5 | 1370 | 6.85E+03 | 34.42 | 33.48 | 33.71 |
| | | stock | | | | | | |
| | | Quantified virus | 22.8 | 456 | 2.28E+03 | 35.16 | 36.24 | 38.27 |
| | | stock | | | | | | |
| | | Quantified virus | 7.6 | 152 | 7.60E+02 | — | 36.94 | — |
| | | stock | | | | | | |

| | | | Detection | | | | | |
|---|---|---|---|---|---|---|---|---|
| | Gene | | limit/PCR | Detection | Detection | PCR | PCR | PCR |
| Virus | target | | reaction | limit/extraction | limit virus/ml | Ct 1 | Ct2 | Ct 3 |
| SARS- | N | Quantified virus | 1000 | 20000 | 1.00E+05 | 27.4 | 27.4 | 27.59 |
| CoV2 | | stock | | | | | | |
| | | Quantified virus | 500 | 10000 | 5.00E+04 | 28.85 | 28.71 | 28.62 |
| | | stock | | | | | | |
| | | Quantified virus | 100 | 2000 | 1.00E+04 | 31.03 | 31.29 | 30.88 |
| | | stock | | | | | | |
| | | Quantified virus | 50 | 1000 | 5.00E+03 | 32.83 | 31.39 | 31.82 |
| | | stock | | | | | | |
| | | Quantified virus | 10 | 200 | 1.00E+03 | 34.79 | 34.20 | 34.15 |
| | | stock | | | | | | |
| | | Quantified virus | 5 | 100 | 5.00E+02 | 38.33 | 34.67 | 36.6 |
| | | stock | | | | | | |
| | | Quantified virus | 1 | 20 | 1.00E+02 | — | | — |
| | | stock | | | | | | |
| Influenza | Matrix | Quantified virus | 1000 | 20000 | 1.00E+05 | 29.03 | 28.82 | 28.78 |
| B | | stock | | | | | | |
| | | Quantified virus | 500 | 10000 | 5.00E+04 | 29.91 | 30.03 | 29.84 |
| | | stock | | | | | | |
| | | Quantified virus | 100 | 2000 | 1.00E+04 | 32.77 | 32.16 | 32.170 |
| | | stock | | | | | | |
| | | Quantified virus | 50 | 1000 | 5.00E+03 | 33.37 | 33.09 | 33.69 |
| | | stock | | | | | | |
| | | Quantified virus | 10 | 200 | 1.00E+03 | 36.12 | 35.33 | 35.95 |
| | | stock | | | | | | |
| | | Quantified virus | 5 | 100 | 5.00E+02 | — | | 38.44 |
| | | stock | | | | | | |
| | | Quantified virus | 1 | 20 | 1.00E+02 | — | — | — |
| | | stock | | | | | | |

Quantified virus stocks were serially diluted, extracted and tested in triplicate with the quadruplex CII-SARS-Cov2 assay. The lowest concentrations detected in all three replicates for each agent are highlighted.

### Example 10—Validation of Quadraplex CII-SARS-CoV-2 rRT-PCR—Cross-Reactivity—Analytical Sensitivity

The assay described in Example 8 was validated for cross-reactivity using in vitro assessment as follows.

Reactivity of the Quadraplex CII-SARS-CoV-2 rRT-PCR assay primer and probe sets were tested in vitro for potential cross-reactivity with sequences of other representative respiratory viral and bacterial pathogen. No amplification was observed with any of the templates, providing no evidence of potential false positive results with the tested organisms (Table 15).

TABLE 15
Specificity testing of Quadruplex CII-SARS-CoV2 assay

| Agent | Source | Concentration | Ct |
|---|---|---|---|
| Influenza A | Clinical specimen | NA | 27.6 |
| Influenza A | Clinical specimen | NA | 17.9 |
| Influenza A | Clinical specimen | NA | 30.9 |
| Influenza A | Clinical specimen | NA | 18.3 |
| Influenza A | Clinical specimen | NA | 18.8 |
| Influenza A | Clinical specimen | NA | 19.7 |
| Human metapneumovirus | Clinical specimen | NA | ND* |
| Human metapneumovirus | Clinical specimen | 3.30E+03 | ND* |
| Human coronavirus OC43 | Clinical specimen | NA | ND* |
| Human parainfluenza virus - 1 | Clinical specimen | NA | ND* |
| Human parainfluenza virus - 3 | Clinical specimen | NA | ND* |
| Human parainfluenza virus - 4 | Clinical specimen | NA | ND* |
| Respiratory syncytial virus B | Clinical specimen | NA | ND* |
| Respiratory syncytial virus B | Clinical specimen | NA | ND* |
| Cytomegalovirus | Clinical specimen | NA | ND* |
| Rhinovirus A | Clinical specimen | NA | ND* |
| Rhinovirus A | Clinical specimen | NA | ND* |
| Rhinovirus C | Clinical specimen | NA | ND* |
| Epstein Barr virus | Clinical specimen | NA | ND* |
| human nasal wash (N = 10) | Clinical specimen | NA | ND* |
| Influenza A virus | virus spiked into | 7.5E+06 | 20.5 |
| (A/California/07/2004(H3N2)) | nasal wash | | |
| Influenza A virus | virus spiked into | 2.5E+08 | 16.0 |
| (A/Brisbane/59/2007(H1N1)) | nasal wash | | |
| Influenza B virus | virus spiked into | 2.0E+08 | 16.60 |
| (B/Shanghai/361/2002) | nasal wash | | |
| Influenza B virus | virus spiked into | 2.1E+08 | 16.91 |
| (B/Yamagata/16/1988) | nasal wash | | |
| Influenza B virus | virus spiked into | 8.0E+07 | 17.52 |
| (B/Malaysia/2506/2004) | nasal wash | | |
| Haemophilus influenzae | ATCC 10211 | NA | ND* |
| Mycobacterium tuberculosis | Clinical specimen | 2.10E+04 | ND* |
| Streptococcus pneumoniae | NR-20805 | 2.80E+05 | ND* |
| Streptococcus pyogenes | NR-50136 | NA | ND* |
| Bordetella pertussis | NR-42457 | 4.32E+05 | ND* |
| Pseudomonas aeruginosa | NR-50573 | NA | ND* |
| Human CoV 229E | ATCC VR-740 | ~1.00E+08 | ND* |
| Human CoV OC43 | ATCC VR-1558 | ~1.00E+05 | ND* |
| Human CoV HKU 1 | ATCC VR-3262SC | ~1.00E+05 | ND* |
| Human CoV NL63 | ATCC VR-3263SD | ~1.00E+05 | ND* |
| Human metapneumovirus (hMPV) | Clinical isolate | 3.30E+03 | ND* |
| Parainfluenza virus 1 | ATCC VR-94 | 4.88E+03 | ND* |
| Parainfluenza virus 2 | ATCC VR-92D | 2.89E+04 | ND* |
| Parainfluenza virus 3 | ATCC VR-1782 | 1.29E+05 | ND* |
| Enterovirus D68 | ATCC VR-1076 | 4.45E+05 | ND* |
| Respiratory syncytial virus (RSV) | ATCC VR-1540 | 1.95E+05 | ND* |
| Rhinovirus | ATCC HRV16 | 5.00E+05 | ND* |
| MERS-CoV | BEI resources | 1.00E+06 | ND* |

*ND—no detection
NA—concentration was not available.

### Example 11—Clinical Evaluation of Quadraplex CII-SARS-CoV-2 rRT-PCR

200 μl from each positive sample was extracted and 100 μl and 5 μl eluted and tested in triplicate using the assay of Example 8. The numbers in Tables 16-18 represent Ct values obtained for each virus primer pair and Rnase P primer pair.

For SARS-CoV-2, 48/48 positive clinical samples using SEQ ID NOs: 1 and 2 were positive (Table 16).

For influenza A, 48/48 positive clinical samples were positive using SEQ ID NOs: 13 and 14 (Table 17).

For influenza B, 47/49 positive clinical samples were positive using SEQ ID NOs: 116 and 17 (Table 18).

TABLE 16
Quadruplex CII-SARS-CoV2 detection of 48/48
clinical samples positive for SARS-CoV-2

| Sample | | | | | | |
|---|---|---|---|---|---|---|
| number | COVID1 | COVID2 | COVID3 | RNaseP 1 | RNaseP 2 | RNaseP 3 |
| 1 | 19.36 | 19.31 | 19.08 | 27.56 | 27.71 | 27.60 |
| 2 | 27.48 | 27.16 | 26.90 | 26.71 | 26.45 | 26.29 |
| 3 | 19.14 | 19.24 | 18.89 | 23.36 | 23.49 | 23.49 |
| 4 | 18.36 | 18.29 | 18.03 | 31.52 | 31.70 | 31.58 |
| 5 | 17.51 | 17.58 | 17.84 | 31.05 | 31.84 | 33.64 |
| 6 | 17.73 | 17.75 | 17.59 | 35.48 | 35.12 | 35.96 |
| 7 | 24.32 | 24.11 | 24.17 | 26.41 | 26.24 | 26.32 |
| 8 | 17.76 | 17.93 | 17.93 | 34.44 | 37.28 | 35.46 |
| 9 | 15.02 | 14.83 | 14.77 | 27.53 | 27.34 | 26.75 |
| 10 | 21.90 | 21.84 | 21.62 | 28.73 | 28.73 | 28.69 |
| 11 | 22.34 | 22.10 | 22.12 | 26.26 | 26.27 | 26.12 |
| 12 | 25.28 | 25.20 | 25.24 | 31.30 | 31.19 | 31.35 |
| 13 | 20.37 | 20.28 | 20.21 | 30.23 | 30.61 | 30.02 |
| 14 | 20.79 | 20.85 | 20.66 | 27.00 | 26.80 | 26.71 |
| 15 | 20.02 | 20.28 | 19.75 | 33.60 | 32.30 | 32.81 |
| 16 | 18.79 | 18.58 | 18.68 | 32.75 | 31.68 | 31.01 |
| 17 | 20.16 | 19.95 | 20.21 | 23.61 | 23.41 | 23.49 |
| 18 | 23.06 | 22.91 | 23.20 | 26.29 | 26.38 | 26.62 |
| 19 | 33.14 | 33.04 | 33.12 | 28.13 | 27.77 | 27.71 |
| 20 | 31.20 | 31.18 | 31.75 | 26.50 | 26.61 | 26.62 |
| 21 | 15.29 | 15.05 | 15.10 | 34.48 | 32.21 | 32.91 |
| 22 | 16.33 | 16.16 | 16.23 | 38.26 | 37.01 | 37.85 |
| 23 | 20.25 | 20.13 | 20.13 | 31.14 | 30.46 | 31.77 |
| 24 | 16.26 | 16.32 | 16.37 | 41.50 | — | 29.16 |
| 25 | 21.89 | 22.31 | 22.16 | 27.96 | 28.40 | 28.26 |
| 26 | 21.81 | 22.22 | 22.08 | 29.99 | 29.85 | 30.09 |
| 27 | 21.94 | 22.04 | 21.94 | 35.96 | 38.18 | 37.22 |
| 28 | 16.08 | 16.21 | 16.27 | 37.88 | 39.23 | 36.65 |
| 29 | 29.72 | 29.89 | 30.29 | 27.45 | 27.38 | 27.74 |
| 30 | 22.08 | 21.70 | 22.05 | 28.03 | 27.65 | 28.20 |
| 31 | 18.71 | 18.39 | 18.49 | 25.13 | 25.15 | 25.40 |
| 32 | 18.75 | 19.05 | 19.06 | 40.62 | 40.07 | 40.83 |
| 33 | 20.38 | 20.30 | 20.66 | 33.50 | 33.64 | 34.79 |
| 34 | 21.10 | 21.02 | 21.10 | 29.62 | 29.87 | 30.81 |
| 35 | 37.04 | 44.63 | 36.56 | 32.25 | 31.89 | 32.53 |
| 36 | 19.74 | 19.94 | 20.13 | 26.25 | 26.45 | 26.60 |
| 37 | 16.41 | 17.04 | 17.26 | 31.85 | 32.91 | 34.19 |
| 38 | 29.82 | 29.77 | 30.31 | 27.55 | 27.50 | 28.10 |
| 39 | 26.10 | 26.82 | 27.04 | 29.95 | 30.51 | 30.86 |
| 40 | 22.33 | 22.75 | 22.76 | 26.70 | 26.97 | 27.12 |
| 41 | 24.49 | 24.60 | 25.03 | 25.66 | 25.68 | 26.39 |
| 42 | 19.29 | 19.41 | 19.31 | 29.99 | 31.10 | 29.59 |
| 43 | 29.18 | 29.09 | 29.07 | 30.11 | 29.92 | 29.67 |
| 44 | 32.40 | 31.84 | 32.17 | 30.00 | 30.02 | 30.15 |
| 45 | 16.42 | 16.81 | 17.05 | 31.07 | 30.98 | 31.20 |
| 46 | 18.51 | 18.60 | 18.97 | 24.59 | 24.72 | 24.90 |
| 47 | 20.21 | 20.44 | 20.90 | 26.79 | 27.05 | 27.11 |
| 48 | 18.35 | 18.19 | 18.35 | 28.80 | 28.97 | 29.01 |

TABLE 17
Quadruplex CII-SARS-CoV2 detection of 48/48
clinical samples positive for influenza A

| Sample | | | | | | |
|---|---|---|---|---|---|---|
| number | FluA 1 | FluA 2 | FluA 3 | RNaseP 1 | RNaseP 2 | RNaseP 3 |
| 1 | 32.89 | 33.27 | 33.44 | 21.67 | 22.30 | 22.20 |
| 2 | 19.09 | 19.05 | 19.00 | 26.63 | 26.61 | 26.35 |
| 3 | 26.26 | 27.03 | 27.04 | 31.96 | 32.09 | 31.80 |
| 4 | 20.90 | 21.06 | 21.51 | 27.38 | 27.49 | 28.05 |
| 5 | 23.15 | 23.21 | 23.46 | 24.26 | 24.26 | 24.47 |
| 6 | 25.14 | 25.18 | 24.94 | 25.90 | 25.81 | 25.47 |
| 7 | 26.09 | 25.86 | 26.03 | 27.45 | 27.55 | 27.41 |
| 8 | 23.82 | 23.81 | 23.68 | 24.64 | 24.91 | 24.61 |
| 9 | 29.49 | 29.36 | 30.31 | 30.42 | 30.17 | 31.28 |
| 10 | 17.49 | 17.51 | 17.80 | 24.52 | 24.60 | 24.95 |
| 11 | 22.98 | 22.61 | 22.91 | 25.90 | 25.76 | 25.87 |
| 12 | 21.15 | 21.36 | 21.43 | 25.51 | 25.81 | 25.88 |
| 13 | 21.32 | 21.27 | 21.50 | 25.78 | 25.77 | 26.04 |
| 14 | 25.72 | 25.87 | 25.93 | 25.23 | 25.27 | 25.18 |
| 15 | 35.80 | 35.47 | 35.33 | 25.13 | 24.69 | 25.02 |
| 16 | 24.48 | 24.32 | 24.52 | 26.93 | 26.90 | 27.03 |
| 17 | 24.09 | 23.61 | 23.91 | 27.91 | 27.42 | 27.65 |
| 18 | 23.39 | 23.31 | 23.61 | 26.25 | 26.11 | 26.32 |
| 19 | 20.52 | 20.30 | 20.47 | 25.58 | 25.37 | 25.88 |
| 20 | 20.85 | 20.76 | 20.92 | 25.72 | 25.56 | 25.95 |
| 21 | 20.24 | 20.20 | 22.34 | 24.66 | 24.51 | 26.65 |
| 22 | 30.31 | 30.27 | 30.32 | 28.13 | 28.02 | 28.07 |
| 23 | 20.15 | 20.13 | 20.25 | 24.74 | 24.89 | 24.86 |
| 24 | 23.71 | 23.60 | 24.54 | 27.30 | 27.23 | 28.18 |
| 25 | 23.92 | 23.98 | 24.41 | 27.21 | 27.15 | 27.82 |
| 26 | 23.26 | 23.43 | 23.77 | 28.28 | 28.27 | 28.52 |
| 27 | 21.68 | 21.92 | 22.01 | 23.72 | 23.88 | 24.09 |
| 28 | 21.90 | 22.16 | 22.50 | 24.26 | 24.15 | 24.99 |
| 29 | 26.03 | 26.02 | 26.18 | 24.77 | 24.89 | 24.99 |
| 30 | 37.45 | 35.49 | 36.54 | 24.10 | 24.44 | 24.42 |
| 31 | 24.62 | 24.68 | 25.56 | 23.15 | 23.29 | 24.26 |
| 32 | 24.76 | 25.26 | 25.28 | 24.03 | 24.39 | 24.56 |
| 33 | 31.98 | 32.17 | 32.69 | 25.43 | 25.96 | 25.63 |
| 34 | 21.01 | 21.10 | 20.86 | 25.33 | 25.46 | 25.27 |
| 35 | 19.61 | 19.94 | 20.30 | 26.66 | 27.09 | 27.63 |
| 36 | 30.80 | 31.33 | 31.41 | 24.88 | 25.22 | 25.29 |
| 37 | 25.15 | 25.45 | 25.26 | 24.13 | 24.58 | 24.12 |
| 38 | 22.75 | 23.81 | 23.56 | 24.22 | 25.24 | 24.92 |
| 39 | 25.94 | 26.09 | 26.42 | 28.98 | 29.16 | 29.36 |
| 40 | 25.37 | 25.86 | 26.26 | 26.03 | 26.47 | 26.84 |
| 41 | 25.93 | 26.13 | 26.63 | 26.85 | 27.14 | 27.68 |
| 42 | 18.36 | 18.36 | 18.29 | 25.19 | 25.10 | 25.17 |
| 43 | 26.77 | 27.09 | 26.89 | 25.34 | 25.70 | 25.47 |
| 44 | 21.76 | 22.81 | 23.33 | 25.55 | 26.46 | 27.14 |
| 45 | 33.42 | 33.87 | 33.67 | 27.23 | 27.72 | 27.42 |
| 46 | 22.69 | 22.71 | 22.81 | 26.68 | 26.87 | 26.74 |
| 47 | 26.94 | 26.84 | 26.94 | 26.83 | 26.60 | 26.82 |
| 48 | 30.69 | 30.60 | 30.73 | 27.18 | 27.29 | 27.48 |

TABLE 18
Quadruplex CII-SARS-CoV2 detection of 47/49 clinical
samples positive for Influenza B virus.

| Sample | | | | | | |
|---|---|---|---|---|---|---|
| number | FluB 1 | FluB 2 | FluB 3 | RNaseP 1 | RNaseP 2 | RNaseP 3 |
| 1 | 22.82 | 22.76 | 22.96 | 20.44 | 20.39 | 20.44 |
| 2 | 25.85 | 25.67 | 25.85 | 27.85 | 27.54 | 27.62 |
| 3 | 14.97 | 14.97 | 15.26 | 25.81 | 25.65 | 25.97 |
| 4 | 25.79 | 25.01 | 24.96 | 29.29 | 28.68 | 28.53 |
| 5 | 20.54 | 20.84 | 20.71 | 23.43 | 23.69 | 23.64 |
| 6 | 23.11 | 23.36 | 23.50 | 27.61 | 27.89 | 27.76 |
| 8 | 26.16 | 26.16 | 26.15 | 28.22 | 28.17 | 28.03 |
| 9 | 27.23 | 27.24 | 27.09 | 23.04 | 22.83 | 22.94 |
| 10 | 35.98 | 37.58 | 37.56 | 26.46 | 26.28 | 26.54 |
| 11 | — | 39.14 | 39.54 | 28.78 | 30.21 | 28.82 |
| 12 | 27.31 | 27.27 | 27.03 | 24.32 | 24.38 | 24.28 |
| 13 | 26.38 | 26.14 | 26.21 | 25.41 | 25.29 | 25.28 |
| 14 | 25.24 | 25.01 | 25.20 | 25.47 | 25.25 | 25.48 |
| 15 | 32.14 | 32.12 | 31.78 | 24.95 | 25.01 | 25.02 |
| 16 | 23.61 | 23.62 | 23.54 | 27.17 | 27.27 | 27.26 |
| 17 | 21.83 | 21.74 | 22.21 | 29.25 | 29.33 | 29.33 |
| 18 | 23.40 | 23.44 | 23.72 | 22.18 | 22.20 | 22.30 |
| 19 | 23.71 | 23.83 | 23.81 | 26.37 | 26.64 | 26.53 |
| 20 | 28.68 | 29.05 | 29.08 | 28.11 | 28.01 | 28.24 |
| 21 | 27.05 | 26.61 | 26.94 | 27.98 | 27.63 | 27.81 |
| 22 | 26.72 | 26.63 | 26.75 | 27.44 | 27.34 | 27.36 |
| 23 | 22.33 | 22.24 | 22.36 | 25.66 | 25.73 | 25.68 |
| 24 | 26.20 | 25.92 | 26.19 | 26.74 | 26.72 | 26.81 |
| 25 | 26.48 | 26.45 | 26.42 | 27.82 | 27.58 | 27.74 |
| 26 | 20.58 | 20.68 | 21.01 | 26.23 | 26.15 | 26.25 |
| 27 | 30.21 | 30.07 | 30.34 | 25.15 | 25.44 | 25.22 |
| 28 | 24.33 | 24.29 | 24.32 | 26.92 | 26.88 | 26.99 |
| 29 | 19.71 | 20.03 | 19.81 | 25.06 | 25.15 | 25.08 |
| 30 | 20.00 | 19.81 | 20.00 | 28.16 | 28.15 | 28.23 |
| 31 | 38.22 | — | — | 26.51 | 26.53 | 26.65 |
| 32 | 30.61 | 30.57 | 30.67 | 23.58 | 23.84 | 23.89 |
| 33 | 23.20 | 23.17 | 23.23 | 24.82 | 24.84 | 24.86 |
| 34 | 26.35 | 26.57 | 26.24 | 23.77 | 24.04 | 23.82 |
| 35 | 21.32 | 21.20 | 21.29 | 26.29 | 26.24 | 26.25 |
| 36 | 24.68 | 24.61 | 24.42 | 24.44 | 24.41 | 24.37 |
| 37 | 33.67 | 33.98 | 33.73 | 28.17 | 28.19 | 28.17 |
| 38 | 19.77 | 19.67 | 19.80 | 27.55 | 27.58 | 27.58 |
| 39 | 37.28 | 38.99 | 36.78 | 27.63 | 27.82 | 27.90 |
| 40 | 35.03 | 35.79 | 36.53 | 26.86 | 26.95 | 26.85 |
| 41 | 23.01 | 22.85 | 23.04 | 27.22 | 27.21 | 27.29 |
| 42 | 24.44 | 24.34 | 24.41 | 27.45 | 27.40 | 27.37 |
| 43 | 27.69 | 27.52 | 27.34 | 27.32 | 27.11 | 27.07 |
| 44 | 18.31 | 18.33 | 18.27 | 26.58 | 26.52 | 26.43 |
| 45 | 30.99 | 31.05 | 31.32 | 26.68 | 26.91 | 26.78 |
| 46 | 22.23 | 22.22 | 22.09 | 31.41 | 31.73 | 31.68 |
| 47 | 34.68 | 34.23 | 34.31 | 30.29 | 30.13 | 30.00 |
| 48 | 23.28 | 23.19 | 23.33 | 25.01 | 24.88 | 25.05 |
| 49 | 22.21 | 22.13 | 22.19 | 25.41 | 25.38 | 25.49 |

1. A method for detecting the presence of the SARS-CoV-2 virus, in a sample comprising:
a. contacting the sample with at least one primer specific the SARS-CoV-2 virus;
b. subjecting the sample and the primers to amplification conditions;
c. detecting the presence of amplification product, wherein the presence of amplification product from the primer specific to SARS-CoV-2 virus indicates the presence of nucleic acid from SARS-CoV-2 virus in the sample.

2. The method of claim 1, wherein the primer comprises the nucleotide sequence chosen from the group consisting of SEQ ID NOs: 1, 2, 4, 5, 7, 8, 10, 11, and combinations thereof.

3. The method of claim 1, further comprising contacting the sample with a probe specific for the SARS-CoV-2 virus.

4. The method of claim 3, wherein the probe has the nucleotide sequence chosen from the group consisting of SEQ ID NOs: 3, 6, 9, and 12.

5. The method of claim 1, wherein the sample is of natural origin and is chosen from the group consisting of nasal swabs, nasopharyngeal aspirates, oropharyngeal aspirates. feces, saliva, plasma, serum, whole blood, spinal fluid, semen, amniotic fluid, lymph fluid, synovial fluid, urine, tears, blood cells, organs, and tissue.

6. The method of claim 1, wherein the sample is chosen from the group consisting of feces, nasopharyngeal aspirates oropharyngeal aspirates, saliva and nasal swabs.

7. The method of claim 1, wherein the sample is from a human subject.

8. A method of detecting the presence of the SARS-CoV-2 virus and at least one other virus chosen from the group consisting of influenza A and influenza B, in a sample comprising:
a. contacting the sample with at least one primer specific the SARS-CoV-2 virus and at least one primer specific from another virus chosen from the group consisting of influenza A and influenza B;
b. subjecting the sample and the primers to amplification conditions;
c. detecting the presence of amplification product, wherein the presence of amplification product from the primer specific to SARS-CoV-2 virus indicates the presence of nucleic acid from SARS-CoV-2 virus in the sample, and the presence of amplification product from the primer specific for the at least one virus chosen from the group consisting of influenza A and influenza B indicates the presence of nucleic acid from that virus in the sample.

9. The method of claim 8, further comprising contacting the sample with a probe specific for SARS-CoV-2 virus and the at least one other virus chosen from the group consisting of influenza A and influenza B.

10. The method of claim 8, wherein the sample is of natural origin and is chosen from the group consisting of nasal swabs, nasopharyngeal aspirates, oropharyngeal aspirates, feces, saliva, plasma, serum, whole blood, spinal fluid, semen, amniotic fluid, lymph fluid, synovial fluid, urine, tears, blood cells, organs, and tissue.

11. The method of claim 8, wherein the sample the sample is chosen from the group consisting of feces, nasopharyngeal aspirates oropharyngeal aspirates, saliva and nasal swabs.

12. The method of claim 8, wherein the sample is from a human subject.

13. The method of claim 8, wherein the primer specific for SARS-CoV-2 comprise the nucleotide sequence chosen from the group consisting of SEQ ID NOs: 1, 2, 4, 5, 7, 8, 10, 11, and combinations thereof.

14. The method of claim 9, wherein the probe specific for SARS-CoV-2 comprises the nucleotide sequence chosen from the group consisting of SEQ ID NOs: 3, 6, 9, and 12.

15. The method of claim 9, wherein the primers specific for SARS-CoV-2 comprise SEQ ID NOs: 1 and 2, and the probe comprises SEQ ID NO: 3.

16. The method of claim 9, wherein the primers specific for influenza A comprise SEQ ID NOs: 4 and 5, and the probe comprises SEQ ID NO: 6.

17. The method of claim 9, wherein the primers specific for influenza B comprise SEQ ID NOs: 7 and 8, and the probe comprises SEQ ID NO: 9.

18. A method of simultaneously detecting and differentiating the presence of SARS-CoV-2 virus and influenza A and influenza B, in a sample comprising:
a. contacting the sample with first primers chosen from the group consisting of SEQ ID NOs: 1, 13, and 16;
b. further contacting the sample with second primers chosen from the group consisting of SEQ ID NOs: 2, 14, and 17;
c. subjecting the sample and the primers to amplification conditions; and
d. detecting the presence of amplification product, wherein the presence of amplification product from primers SEQ ID NOs: 1 and 2 indicates the presence of nucleic acid from the SARS-CoV-2 virus in the sample, the presence of amplification product from primers SEQ ID NOs: 13 and 14 indicates the presence of nucleic acid from influenza A in the sample, and the presence of amplification product from primers SEQ ID NOs: 16 and 17 indicates the presence of nucleic acid from influenza B, and wherein the primers SEQ ID NOs: 1, 2, 13, 14, 16, and 17, allow the differential detection of each virus in the sample;
or
a method of detecting the presence of SARS-CoV-2 virus, in a sample comprising:
a. contacting the sample with first primers comprising SEQ ID NOs: 1 and 4;
b. further contacting the sample with second primers comprising SEQ ID NOs: 2 and 5.
c. subjecting the sample and the primers to amplification conditions; and
d. detecting the presence of amplification product, wherein the presence of amplification product from any pair of primers indicates the presence of nucleic acid from the SARS-CoV-2 virus in the sample.

19. The method of claim 18, further comprising contacting the sample with probes comprising SEQ ID NOs: 3, 15, and 18.

20. The method of claim 19, wherein the probes are detectably labeled.

21-34. (canceled)
